SEQUENCE LISTING (16 sequences)

SEQ ID NO: 1 - DNA, 18 bp, Artificial Sequence
  Oligonucleotide Primer
acccatcaccatcttcca

SEQ ID NO: 2 - DNA, 19 bp, Artificial Sequence
  Oligonucleotide Primer
catcacgccacagctttcc

SEQ ID NO: 3 - DNA, 20 bp, Artificial Sequence
  Oligonucleotide Primer
agtcgcgttctggagaggta

SEQ ID NO: 4 - DNA, 20 bp, Artificial Sequence
  Oligonucleotide Primer
aggatggcttctgagagcag

SEQ ID NO: 5 - protein, 826 aa, Homo Sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 6 - DNA, 3958 bp, Homo Sapiens
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggaaaaaaaaaaaaaaaaaaa

SEQ ID NO: 7 - protein, 735 aa, Homo Sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGII

SEQ ID NO: 8 - DNA, 3812 bp, Homo Sapiens
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 9 - protein, 870 aa, Homo Sapiens
MTADKEKKRSSSERRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASRLLGPSFESYLLPELTRYDCEVNVPVLGSSTLLQGGDLLRALDQAT

SEQ ID NO: 10 - DNA, 5186 bp, Homo Sapiens
gccacacgggtccggtgcccgctgcgcttccgccccagcgctcctgaggcggccgtacaatcctcggcagtgtcctgagactgtatggtcagctcagcccggcctccgactccttccgactcccagcattcgagccacttttttttttctttgaaaactcagaaaagtgactccttttccagggaaaaaggaacttgggttcccttctctccgtcctcttttcgggtctgacagcctccacccactccttccccggaccccgcctccgcgcgcaggttcctcccagtcacctttctccacccccgcccccgcacctagcccgccgcgcgccaccttccacctgactgcgcggggcgctcgggacctgcgcgcacctcggaccttcaccacccgcccgggccgcggggagcggacgagggccacagccccccacccgccagggagcccaggtgctcggcgtctgaacgtctcaaagggccacagcgacaatgacagctgacaaggagaagaaaaggagtagctcggagaggaggaaggagaagtcccgggatgctgcgcggtgccggcggagcaaggagacggaggtgttctatgagctggcccatgagctgcctctgccccacagtgtgagctcccatctggacaaggcctccatcatgcgactggcaatcagcttcctgcgaacacacaagctcctctcctcagtttgctctgaaaacgagtccgaagccgaagctgaccagcagatggacaacttgtacctgaaagccttggagggtttcattgccgtggtgacccaagatggcgacatgatctttctgtcagaaaacatcagcaagttcatgggacttacacaggtggagctaacaggacatagtatctttgacttcactcatccctgcgaccatgaggagattcgtgagaacctgagtctcaaaaatggctctggttttgggaaaaaaagcaaagacatgtccacagagcgggacttcttcatgaggatgaagtgcacggtcaccaacagaggccgtactgtcaacctcaagtcagccacctggaaggtcttgcactgcacgggccaggtgaaagtctacaacaactgccctcctcacaatagtctgtgtggctacaaggagcccctgctgtcctgcctcatcatcatgtgtgaaccaatccagcacccatcccacatggacatccccctggatagcaagaccttcctgagccgccacagcatggacatgaagttcacctactgtgatgacagaatcacagaactgattggttaccaccctgaggagctgcttggccgctcagcctatgaattctaccatgcgctagactccgagaacatgaccaagagtcaccagaacttgtgcaccaagggtcaggtagtaagtggccagtaccggatgctcgcaaagcatgggggctacgtgtggctggagacccaggggacggtcatctacaaccctcgcaacctgcagccccagtgcatcatgtgtgtcaactacgtcctgagtgagattgagaagaatgacgtggtgttctccatggaccagactgaatccctgttcaagccccacctgatggccatgaacagcatctttgatagcagtggcaagggggctgtgtctgagaagagtaacttcctattcaccaagctaaaggaggagcccgaggagctggcccagctggctcccaccccaggagacgccatcatctctctggatttcgggaatcagaacttcgaggagtcctcagcctatggcaaggccatcctgcccccgagccagccatgggccacggagttgaggagccacagcacccagagcgaggctgggagcctgcctgccttcaccgtgccccaggcagctgccccgggcagcaccacccccagtgccaccagcagcagcagcagctgctccacgcccaatagccctgaagactattacacatctttggataacgacctgaagattgaagtgattgagaagctcttcgccatggacacagaggccaaggaccaatgcagtacccagacggatttcaatgagctggacttggagacactggcaccctatatccccatggacggggaagacttccagctaagccccatctgccccgaggagcggctcttggcggagaacccacagtccaccccccagcactgcttcagtgccatgacaaacatcttccagccactggcccctgtagccccgcacagtcccttcctcctggacaagtttcagcagcagctggagagcaagaagacagagcccgagcaccggcccatgtcctccatcttctttgatgccggaagcaaagcatccctgccaccgtgctgtggccaggccagcacccctctctcttccatggggggcagatccaatacccagtggcccccagatccaccattacattttgggcccacaaagtgggccgtcggggatcagcgcacagagttcttgggagcagcgccgttggggccccctgtctctccaccccatgtctccaccttcaagacaaggtctgcaaagggttttggggctcgaggcccagacgtgctgagtccggccatggtagccctctccaacaagctgaagctgaagcgacagctggagtatgaagagcaagccttccaggacctgagcgggggggacccacctggtggcagcacctcacatttgatgtggaaacggatgaagaacctcaggggtgggagctgccctttgatgccggacaagccactgagcgcaaatgtacccaatgataagttcacccaaaaccccatgaggggcctgggccatcccctgagacatctgccgctgccacagcctccatctgccatcagtcccggggagaacagcaagagcaggttccccccacagtgctacgccacccagtaccaggactacagcctgtcgtcagcccacaaggtgtcaggcatggcaagccggctgctcgggccctcatttgagtcctacctgctgcccgaactgaccagatatgactgtgaggtgaacgtgcccgtgctgggaagctccacgctcctgcaaggaggggacctcctcagagccctggaccaggccacctgagccaggccttctacctgggcagcacctctgccgacgccgtcccaccagcttcactctctccgtctgtttttgcaactaggtatttctaacgccagcacactatttacaagatggacttacctggcagacttgcccaggtcaccaagcagtggcctttttctgagatgctcactttattatccctatttttaaagtacacaattgttttacctgttctgaaatgttcttaaattttgtaggatttttttcctccccaccttcaatgacttctaatttatattatccataggtttctctccctccttctccttctcacacacaactgtccatactaacaagtttggtgcatgtctgttcttctgtagggagaagctttagcttcattttactaaaaagattcctcgttattgttgttgccaaagagaaacaaaaatgattttgctttccaagcttggtttgtggcgtctccctcgcagagcccttctcgtttcttttttaaactaatcaccatattgtaaatttcagggtttttttttttttgtttaagctgactctttgctctaattttggaaaaaaagaaatgtgaagggtcaactccaacgtatgtggttatctgtgaaagttgcacagcgtggcttttcctaaactggtgtttttcccccgcatttggtggattttttattattattcaaaaacataactgagttttttaaaagaggagaaaatttatatctgggttaagtgtttatcatatatatgggtactttgtaatatctaaaaacttagaaacggaaatggaatcctgctcacaaaatcactttaagatcttttcgaagctgttaatttttcttagtgttgtggacactgcagacttgtccagtgctcccacggcctgtacggacactgtggaaggcctccctctgtcggctttttgccatctgtgatatgccataggtgtgacaatccgagcagtggagtcattcagcgggagcactgcgcgctatcccctcacattctctatgtactatgtatgtatgtattattattattgctgccaagagggtctgatggcacgttgtggggtcggggggtggggcggggaagtgctctaacttttcttaaggttttgttgctagcccttcaagtgcactgagctatgtgactcggatggtctttcacacggcacatttggacatttccagaactaccatgagatggtttagacgggaattcatgcaaatgaggggtcaaaaatggtatagtgaccccgtccacgtcctccaagctcacgaccttggagccccgtggagctggactgaggaggaggctgcacagcgggagagcagctggtccagaccagccctgcagcccccactcagccggcagccagatggccccgcaaggcctccagggatggcccctagccacaggccctggctgaggtctctgggtcggtcagtgacatgtaggtaggaagcactgaaaatagtgttcccagagcactttgcaactccctgggtaagagggacgacacctctggtttttcaataccaattacatggaacttttctgtaatgggtacaatgaagaagtttctaaaaacacacacaaagcacattgggccaactatttagtaagcccggatagacttattgccaaaaacaaaaaatagctttcaaaagaaatttaagttctatgagaaattccttagtcatggtgttgcgtaaatcatattttagctgcacggcattaccccacacagggtggcagaacttgaagggttactgacgtgtaaatgctggtatttgatttcctgtgtgtgttgccctggcattaagggcattttacccttgcagttttactaaaacactgaaaaatattccaagcttcatattaaccctacctgtcaacgtaacgatttcatgaacgttattatattgtcgaattcctactgacaacattataactgtatgggagcttaactttataaggaaatgtattttgacactggtatcttattaaagtattctgatcctaaaaaaaaaaaaaaaaaaaaaaaaaaaaaaaaaaaaaaaaaaaaaaaaa

SEQ ID NO: 11 - protein, 856 aa, Homo Sapiens
MTADKEKKRSSSERRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASRLLGPSFESYLLPELTRYDCEVNVPVLGSSTL

SEQ ID NO: 12 - DNA, 5011 bp, Homo Sapiens
cttttccagggaaaaaggaacttgggttcccttctctccgtcctcttttcgggtctgacagcctccacccactccttccccggaccccgcctccgcgcgcaggttcctcccagtcacctttctccacccccgcccccgcacctagcccgccgcgcgccaccttccacctgactgcgcggggcgctcgggacctgcgcgcacctcggaccttcaccacccgcccgggccgcggggagcggacgagggccacagccccccacccgccagggagcccaggtgctcggcgtctgaacgtctcaaagggccacagcgacaatgacagctgacaaggagaagaaaaggagtagctcggagaggaggaaggagaagtcccgggatgctgcgcggtgccggcggagcaaggagacggaggtgttctatgagctggcccatgagctgcctctgccccacagtgtgagctcccatctggacaaggcctccatcatgcgactggcaatcagcttcctgcgaacacacaagctcctctcctcagtttgctctgaaaacgagtccgaagccgaagctgaccagcagatggacaacttgtacctgaaagccttggagggtttcattgccgtggtgacccaagatggcgacatgatctttctgtcagaaaacatcagcaagttcatgggacttacacaggtggagctaacaggacatagtatctttgacttcactcatccctgcgaccatgaggagattcgtgagaacctgagtctcaaaaatggctctggttttgggaaaaaaagcaaagacatgtccacagagcgggacttcttcatgaggatgaagtgcacggtcaccaacagaggccgtactgtcaacctcaagtcagccacctggaaggtcttgcactgcacgggccaggtgaaagtctacaacaactgccctcctcacaatagtctgtgtggctacaaggagcccctgctgtcctgcctcatcatcatgtgtgaaccaatccagcacccatcccacatggacatccccctggatagcaagaccttcctgagccgccacagcatggacatgaagttcacctactgtgatgacagaatcacagaactgattggttaccaccctgaggagctgcttggccgctcagcctatgaattctaccatgcgctagactccgagaacatgaccaagagtcaccagaacttgtgcaccaagggtcaggtagtaagtggccagtaccggatgctcgcaaagcatgggggctacgtgtggctggagacccaggggacggtcatctacaaccctcgcaacctgcagccccagtgcatcatgtgtgtcaactacgtcctgagtgagattgagaagaatgacgtggtgttctccatggaccagactgaatccctgttcaagccccacctgatggccatgaacagcatctttgatagcagtggcaagggggctgtgtctgagaagagtaacttcctattcaccaagctaaaggaggagcccgaggagctggcccagctggctcccaccccaggagacgccatcatctctctggatttcgggaatcagaacttcgaggagtcctcagcctatggcaaggccatcctgcccccgagccagccatgggccacggagttgaggagccacagcacccagagcgaggctgggagcctgcctgccttcaccgtgccccaggcagctgccccgggcagcaccacccccagtgccaccagcagcagcagcagctgctccacgcccaatagccctgaagactattacacatctttggataacgacctgaagattgaagtgattgagaagctcttcgccatggacacagaggccaaggaccaatgcagtacccagacggatttcaatgagctggacttggagacactggcaccctatatccccatggacggggaagacttccagctaagccccatctgccccgaggagcggctcttggcggagaacccacagtccaccccccagcactgcttcagtgccatgacaaacatcttccagccactggcccctgtagccccgcacagtcccttcctcctggacaagtttcagcagcagctggagagcaagaagacagagcccgagcaccggcccatgtcctccatcttctttgatgccggaagcaaagcatccctgccaccgtgctgtggccaggccagcacccctctctcttccatggggggcagatccaatacccagtggcccccagatccaccattacattttgggcccacaaagtgggccgtcggggatcagcgcacagagttcttgggagcagcgccgttggggccccctgtctctccaccccatgtctccaccttcaagacaaggtctgcaaagggttttggggctcgaggcccagacgtgctgagtccggccatggtagccctctccaacaagctgaagctgaagcgacagctggagtatgaagagcaagccttccaggacctgagcgggggggacccacctggtggcagcacctcacatttgatgtggaaacggatgaagaacctcaggggtgggagctgccctttgatgccggacaagccactgagcgcaaatgtacccaatgataagttcacccaaaaccccatgaggggcctgggccatcccctgagacatctgccgctgccacagcctccatctgccatcagtcccggggagaacagcaagagcaggttccccccacagtgctacgccacccagtaccaggactacagcctgtcgtcagcccacaaggtgtcaggcatggcaagccggctgctcgggccctcatttgagtcctacctgctgcccgaactgaccagatatgactgtgaggtgaacgtgcccgtgctgggaagctccacgctcctgcaaggaggggacctcctcagagccctggaccaggccacctgagccaggccttctacctgggcagcacctctgccgacgccgtcccaccagcttcactctctccgtctgtttttgcaactaggtatttctaacgccagcacactatttacaagatggacttacctggcagacttgcccaggtcaccaagcagtggcctttttctgagatgctcactttattatccctatttttaaagtacacaattgttttacctgttctgaaatgttcttaaattttgtaggatttttttcctccccaccttcaatgacttctaatttatattatccataggtttctctccctccttctccttctcacacacaactgtccatactaacaagtttggtgcatgtctgttcttctgtagggagaagctttagcttcattttactaaaaagattcctcgttattgttgttgccaaagagaaacaaaaatgattttgctttccaagcttggtttgtggcgtctccctcgcagagcccttctcgtttcttttttaaactaatcaccatattgtaaatttcagggtttttttttttgtttaagctgactctttgctctaattttggaaaaaaagaaatgtgaagggtcaactccaacgtatgtggttatctgtgaaagttgcacagcgtggcttttcctaaactggtgtttttcccccgcatttggtggattttttattattattcaaaaacataactgagttttttaaaagaggagaaaatttatatctgggttaagtgtttatcatatatatgggtactttgtaatatctaaaaacttagaaacggaaatggaatcctgctcacaaaatcactttaagatcttttcgaagctgttaatttttcttagtgttgtggacactgcagacttgtccagtgctcccacggcctgtacggacactgtggaaggcctccctctgtcggctttttgccatctgtgatatgccataggtgtgacaatccgagcagtggagtcattcagcgggagcactgcgcgctatcccctcacattctctatgtactatgtatgtatgtattattattattgctgccaagagggtctgatggcacgttgtggggtcggggggtggggcggggaagtgctctaacttttcttaaggttttgttgctagcccttcaagtgcactgagctatgtgactcggatggtctttcacacggcacatttggacatttccagaactaccatgagatggtttagacgggaattcatgcaaatgaggggtcaaaaatggtatagtgaccccgtccacgtcctccaagctcacgaccttggagccccgtggagctggactgaggaggaggctgcacagcgggagagcagctggtccagaccagccctgcagcccccactcagccggcagccagatggccccgcaaggcctccagggatggcccctagccacaggccctggctgaggtctctgggtcggtcagtgacatgtaggtaggaagcactgaaaatagtgttcccagagcactttgcaactccctgggtaagagggacgacacctctggtttttcaataccaattacatggaacttttctgtaatgggtacaatgaagaagtttctaaaaacacacacaaagcacattgggccaactatttagtaagcccggatagacttattgccaaaaacaaaaaatagctttcaaaagaaatttaagttctatgagaaattccttagtcatggtgttgcgtaaatcatattttagctgcacggcattaccccacacagggtggcagaacttgaagggttactgacgtgtaaatgctggtatttgatttcctgtgtgtgttgccctggcattaagggcattttacccttgcagttttactaaaacactgaaaaatattccaagcttcatattaaccctacctgtcaacgtaacgatttcatgaacgttattatattgtcgaattcctactgacaacattataactgtatgggagcttaactttataaggaaatgtattttgacactggtatcttattaaagtattctgatcctaaaaaaaaaaaaaaaaaaaaaaaaaaaaaaaaaaaaaaaaaaaaaaaaa

SEQ ID NO: 13 - protein, 870 aa, Homo Sapiens
MTADKEKKRSSSERRKEKSRDAARCRRSKETEVFYELAHELPLPHSVSSHLDKASIMRLEISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASRLLGPSFESYLLPELTRYDCEVNVPVLGSSTLLQGGDLLRALDQAT

SEQ ID NO: 14 - DNA, 2818 bp, Homo Sapiens
cctgactgcgcggggcgctcgggacctgcgcgcacctcggaccttcaccacccgcccgggccgcggggagcggacgagggccacagccccccacccgccagggagcccaggtgctcggcgtctgaacgtctcaaagggccacagcgacaatgacagctgacaaggagaagaaaaggagtagctcggagaggaggaaggagaagtcccgggatgctgcgcggtgccggcggagcaaggagacggaggtgttctatgagctggcccatgagctgcctctgccccacagtgtgagctcccatctggacaaggcctccatcatgcgactggaaatcagcttcctgcgaacacacaagctcctctcctcagtttgctctgaaaacgagtccgaagccgaagctgaccagcagatggacaacttgtacctgaaagccttggagggtttcattgccgtggtgacccaagatggcgacatgatctttctgtcagaaaacatcagcaagttcatgggacttacacaggtggagctaacaggacatagtatctttgacttcactcatccctgcgaccatgaggagattcgtgagaacctgagtctcaaaaatggctctggttttgggaaaaaaagcaaagacatgtccacagagcgggacttcttcatgaggatgaagtgcacggtcaccaacagaggccgtactgtcaacctcaagtcagccacctggaaggtcttgcactgcacgggccaggtgaaagtctacaacaactgccctcctcacaatagtctgtgtggctacaaggagcccctgctgtcctgcctcatcatcatgtgtgaaccaatccagcacccatcccacatggacatccccctggatagcaagaccttcctgagccgccacagcatggacatgaagttcacctactgtgatgacagaatcacagaactgattggttaccaccctgaggagctgcttggccgctcagcctatgaattctaccatgcgctagactccgagaacatgaccaagagtcaccagaacttgtgcaccaagggtcaggtagtaagtggccagtaccggatgctcgcaaagcatgggggctacgtgtggctggagacccaggggacggtcatctacaaccctcgcaacctgcagccccagtgcatcatgtgtgtcaactacgtcctgagtgagattgagaagaatgacgtggtgttctccatggaccagactgaatccctgttcaagccccacctgatggccatgaacagcatctttgatagcagtggcaagggggctgtgtctgagaagagtaacttcctattcaccaagctaaaggaggagcccgaggagctggcccagctggctcccaccccaggagacgccatcatctctctggatttcgggaatcagaacttcgaggagtcctcagcctatggcaaggccatcctgcccccgagccagccatgggccacggagttgaggagccacagcacccagagcgaggctgggagcctgcctgccttcaccgtgccccaggcagctgccccgggcagcaccacccccagtgccaccagcagcagcagcagctgctccacgcccaatagccctgaagactattacacatctttggataacgacctgaagattgaagtgattgagaagctcttcgccatggacacagaggccaaggaccaatgcagtacccagacggatttcaatgagctggacttggagacactggcaccctatatccccatggacggggaagacttccagctaagccccatctgccccgaggagcggctcttggcggagaacccacagtccaccccccagcactgcttcagtgccatgacaaacatcttccagccactggcccctgtagccccgcacagtcccttcctcctggacaagtttcagcagcagctggagagcaagaagacagagcccgagcaccggcccatgtcctccatcttctttgatgccggaagcaaagcatccctgccaccgtgctgtggccaggccagcacccctctctcttccatggggggcagatccaatacccagtggcccccagatccaccattacattttgggcccacaaagtgggccgtcggggatcagcgcacagagttcttgggagcagcgccgttggggccccctgtctctccaccccatgtctccaccttcaagacaaggtctgcaaagggttttggggctcgaggcccagacgtgctgagtccggccatggtagccctctccaacaagctgaagctgaagcgacagctggagtatgaagagcaagccttccaggacctgagcgggggggacccacctggtggcagcacctcacatttgatgtggaaacggatgaagaacctcaggggtgggagctgccctttgatgccggacaagccactgagcgcaaatgtacccaatgataagttcacccaaaaccccatgaggggcctgggccatcccctgagacatctgccgctgccacagcctccatctgccatcagtcccggggagaacagcaagagcaggttccccccacagtgctacgccacccagtaccaggactacagcctgtcgtcagcccacaaggtgtcaggcatggcaagccggctgctcgggccctcatttgagtcctacctgctgcccgaactgaccagatatgactgtgaggtgaacgtgcccgtgctgggaagctccacgctcctgcaaggaggggacctcctcagagccctggaccaggccacctgagccaggccttctacctgggcagcacctctgccgacgccgtcccaccagcttcaccc

SEQ ID NO: 15 - DNA, 20 bp, Artificial Sequence
  Oligonucleotide Primer
tcaagtcagcaacgtggaag

SEQ ID NO: 16 - DNA, 20 bp, Artificial Sequence
  Oligonucleotide Primer
tatcgaggctgggtcgactg

(19) United States
(12) Patent Application Publication
(10) Pub. No.: US 2009/0252814 A1
(43) Pub. Date: Oct. 8, 2009

(54) USE OF XENON FOR ORGAN PROTECTION

(76) Inventors: Mervyn Maze, London (GB); Patrick Henry Maxwell, Oxford (GB); David Edwards, London (GB)

Correspondence Address: ELMORE PATENT LAW GROUP, PC, 515 Groton Road, Unit 1R, Westford, MA, 01886, US

(21) Appl. No.: 12/089,065

(22) PCT Filed: Oct. 3, 2006
(86) PCT No.: PCT/GB2006/003678

§ 371 Date: Nov. 4, 2008

(30) Foreign Application Priority Data
Oct. 4, 2005 (GB) 0520176.9

(51) Int. Cl. A01N 1/02; C12N 5/06; A61K 33/00; C12N 15/85

(52) U.S. Cl. 424/600; 435/1.1; 435/375; 435/1.2; 435/455

(57) ABSTRACT

Use of xenon is described. Xenon is used as an organ and/or tissue and/or cell protectant in the manufacture of a pharmaceutical for the protection from injury of organs and/or tissue and/or cells that express HIF.

### FIELD

The present invention relates to the use of an HIF activator as an organ and/or tissue and/or cell protectant. In particular the present invention relates to the use of xenon, as an HIF activator, in the manufacture of a pharmaceutical for the protection from injury of organs and/or tissue and/or cells that express HIF.

Further, the present invention relates to methods for inducing the expression of HIF and/or at least one downstream effector of HIF in at least one organ and/or tissue and/or cell.

### BACKGROUND

Tissues need a continuous supply of oxygen for effective metabolism. Reduced blood flow (ischaemia) is a common cause of tissue and organ damage. It is now also clear that further damage occurs when flow recommences, probably due to excess generation of reactive oxygen species (ROS) such as H₂O₂—both from resident cells, and from infiltration by activated neutrophils. This is termed ischaemia/reperfusion (I/R) injury, and often damages tissues and organs, e.g., during vascular surgery, heart surgery and in kidney transplantation. Tissue and/or organs are also injured as a result of trauma, or sepsis.

To date several techniques have been used in attempts to protect organs and tissues from injury such as ischaemia/reperfusion—reviewed in Yellon DM and Baxter GF (1999) Reperfusion injury revisited: is there a role for growth factor signalling in limiting lethal reperfusion injury? Trends Cardiovasc Med 9: 245-249. However no pharmacological manipulation has yet been shown to confer clinical benefit when used in this way.

The present invention seeks to overcome some of these problems.

### Broad Aspects

Some of the broad aspects of the present invention are now presented.

In a first broad aspect there is provided the use of an HIF activator as the sole organ and/or tissue and/or cell protectant in the manufacture of a pharmaceutical composition for the protection from injury of organs and/or tissues and/or cells that express HIF, wherein said organ and/or tissue and/or cell is not any of brain or heart; preferably not any of brain or heart, embryonic nigral tissue, liver, lung, cornea, neurones, and endothelial cells of the intestine.

In a second broad aspect there is provided the use of xenon as the sole organ and/or tissue and/or cell protectant in the manufacture of a pharmaceutical composition for the protection from injury of organs and/or tissues and/or cells that express HIF, wherein said organ and/or tissue and/or cell is not any of brain or heart; preferably not any of brain or heart, embryonic nigral tissue, liver, lung, cornea, neurones, and endothelial cells of the intestine.

In a third broad aspect there is provided the use of an HIF activator as the sole organ and/or tissue and/or cell protectant in the manufacture of a pharmaceutical composition for the protection of kidney from injury.

In a fourth broad aspect there is provided the use of xenon as the sole organ and/or tissue and/or cell protectant in the manufacture of a pharmaceutical composition for the protection of kidney from injury.

In a fifth broad aspect there is provided a method of protecting from injury at least one organ and/or tissue and/or cell that expresses HIF; wherein said method comprises the step of administering an HIF activator or a pharmaceutical composition comprising an HIF activator as the sole organ and/or tissue and/or cell protectant to the organ and/or tissue and/or cell, wherein said organ and/or tissue and/or cell is not any of brain or heart; preferably not any of brain or heart, embryonic nigral tissue, liver, lung, cornea, neurones, and endothelial cells of the intestine.

In a sixth broad aspect there is provided a method of protecting from injury at least one organ and/or tissue and/or cell that expresses HIF; wherein said method comprises the step of administering xenon or a pharmaceutical composition comprising xenon as the sole organ and/or tissue and/or cell protectant to the organ and/or tissue and/or cell, wherein said organ and/or tissue and/or cell is not any of brain or heart; preferably not any of brain or heart, embryonic nigral tissue, liver, lung, cornea, neurones, and endothelial cells of the intestine.

In a seventh broad aspect there is provided a method for reducing the expression of at least one upstream degrader of HIF and/or inducing the expression of HIF and/or inducing the expression of at least one downstream effector of HIF in at least one organ and/or tissue and/or cell; wherein said method comprises the step of administering an HIF activator or a composition comprising an HIF activator to said organ and/or tissue and/or cell; wherein said organ and/or tissue and/or cell is not any of brain or heart; preferably not any of brain or heart, embryonic nigral tissue, liver, lung, cornea, neurones, and endothelial cells of the intestine.

In an eighth broad aspect there is provided a method for reducing the expression of an upstream degrader of HIF and/or inducing the expression of HIF and/or inducing the expression of at least one downstream effector of HIF in at least one organ and/or tissue and/or cell; wherein said method comprises the step of administering xenon or a composition comprising xenon to said organ and/or tissue and/or cell; wherein said organ and/or tissue and/or cell is not any of brain or heart; preferably not any of brain or heart, embryonic nigral tissue, liver, lung, cornea, neurones, and endothelial cells of the intestine.

In a ninth broad aspect there is provided the use of xenon as an HIF activator in the manufacture of an organ and/or tissue and/or cell protectant.

In a tenth broad aspect there is provided the use of an HIF activator as the sole organ and/or tissue and/or cell protectant in the manufacture of a pharmaceutical composition for the protection from injury of an organ and/or tissue and/or cell; wherein said pharmaceutical composition is administered before and/or after said organ and/or tissue and/or cell is cooled.

In an eleventh broad aspect there is provided use of xenon as the sole organ and/or tissue and/or cell protectant in the manufacture of a pharmaceutical composition for the protection from injury of an organ and/or tissue and/or cell; wherein said pharmaceutical composition is administered before and/or after said organ and/or tissue and/or cell is cooled.

Preferably the HIF activator is used as an organ and/or tissue protectant.

In these broad aspects preferably the HIF activator is an HIF-1α activator and/or an HIF-2α activator.

More preferably the HIF activator is xenon. Xenon is a chemically inert gas (a noble gas) whose anaesthetic properties have been known for over 50 years (Lawrence J H et al, J. Physiol. 1946; 105:197-204). Since its first use in surgery (Cullen S C et al, Science 1951; 113:580-582), a number of research groups have shown that it has an excellent pharmacological profile, including the absence of metabolic by-products, profound analgesia, rapid onset and recovery, and minimal effects on the cardiovascular system (Lachmann B et al, Lancet 1990; 335:1413-1415; Kennedy R R et al, Anaesth. Intens. Care 1992; 20:66-70; Luttropp H H et al, Acta Anaesthesiol. Scand. 1994; 38:121-125; Goto T et al, Anesthesiology 1997; 86:1273-1278; Marx T et al, Br. J. Anaesth. 1997; 78:326-327).

The exact mechanism of action for the effects of xenon as an anaesthetic is not entirely clear. During recent years a number of studies have elucidated that xenon exhibits effects on the NMDA transmission and xenon has been used as N-methyl-D-aspartate (NMDA) receptor antactivator (see US-B-6,274,633).

The anaesthetic effects of xenon have been claimed to be dose dependent and high concentrations of xenon such as more than 50 vol. % have been suggested to be required for clinical effects. These high concentrations of xenon are associated with profound effects on wakefulness. It is rather clear that humans breathing more than 50 vol. % xenon will enter a light stage of anaesthesia. Mechanistic studies on cultured hippocampal neurons have shown that 80% xenon, which will maintain surgical anaesthesia, reduces NMDA-activated currents by up to 60%. This powerful inhibition of the NMDA receptor explains some of the important features of the pharmacological profile and is likely to be instrumental in the anaesthetic and analgesic effects of this inert gas.

Besides using xenon as an anaesthetic, it has been reported that xenon may provide some cell protecting effects against neurotransmitter excess (see WO-A-00/53192; and Ma et al 2005 Ann Neurol 2005; 58:182-193).

Ma et al (2005; Ann Neurol 2005; 58:182-193) teach that xenon can enhance the neuroprotection provided by mild hypothermia. Ma et al (2005) showed that cultured neurones injured by oxygen-glucose deprivation were protected by combinations of interventions of xenon and hypothermia that, when administered alone, were not efficacious. Furthermore, it was also shown by Ma et al (2005) that a combination of xenon and hypothermia administered 4 hours after hypoxic-ischaemic injury in neonatal rats provided synergistic neuroprotection. Ma et al (2005) suggested that xenon in combination with mild hypothermia may provide a safe and effective therapy for perinatal asphyxia.

It has also been reported that xenon may provide some cell protecting effects against excess release of neurotransmitters—namely neurointoxication—(see, for example, WO00/53192). WO00/53192 teaches that xenon can reduce the release of neurotransmitters, particularly dopamine, which are caused, for example, by hypoxia. Furthermore, WO00/53192 teaches the use of preparations containing xenon for the treatment of depression, schizophrenia and Parkinson's disease.

In addition, it has been reported that xenon administration during early reperfusion reduces infarct size after regional ischaemia in the rabbit heart (Preckel et al., Anesthesia and analgesia, December 2000, 91(6), pages 1327-1332). Furthermore, Weber et al (2005) teach that xenon induces cardioprotection by protein kinase C (PC) and that this cardioprotection is mediated by PKC-ε and its downstream target p38 MAPK. WO00/067945 teaches the use of xenon in combination with carbon monoxide mixture to protect cells (such as those of the heart, brain, kidney or peripheral tissue—POAD) exposed to ischaemia or hypoxia—particularly to protect from ischaemia reperfusion. Carbon monoxide was known, amongst other uses, to improve the outcome of tissue and organ transplants and to suppress apoptosis (WO03/000114).

WO05/039600 teaches the use of xenon or a xenon gas mixture for preventing or reducing cellular death to tissue and organs which are to be transplanted—such as the liver, embryonic nigral tissue and heart. Furthermore, WO05/039600 also teaches the use of xenon for preventing apoptotic cell death after eye laser surgery, and for protecting endothelial cells of the intestine in sepsis.

The cited prior art however does not teach the use of xenon as an HIF activator, let alone an HIF-1α activator and/or HIF-2α activator. Furthermore, the cited prior art does not teach the use of xenon as an HIF activator, in particular an HIF-1α activator and/or HIF-2α activator, as an organ and/or tissue and/or cell protectant.

### Specific Aspects

Specific aspects of the present invention are now presented.

In one aspect of the present invention there is provided the use of xenon as the sole organ and/or tissue and/or cell protectant in the manufacture of a pharmaceutical composition for the protection from injury of organs and/or tissue and/or cells that express HIF, wherein said organ and/or tissue and/or cell is not any of brain, heart, embryonic nigral tissue, liver, lung, cornea, neurones, and endothelial cells of the intestine.

In another aspect of the present invention there is provided the use of xenon as the sole organ and/or tissue and/or cell protectant in the manufacture of a pharmaceutical composition for the protection of kidney from injury.

The present invention provides in another aspect a method of protecting from injury at least one organ and/or tissue and/or cell that expresses HIF; wherein said method comprises the step of administering xenon or a pharmaceutical composition comprising xenon as the sole organ and/or tissue and/or cell protectant to the organ and/or tissue and/or cell wherein said organ and/or tissue and/or cell is not any of brain, heart, embryonic nigral tissue, liver, lung, cornea, neurones, and endothelial cells of the intestine.

In a further aspect the present invention provides a method for reducing the expression of at least one upstream degrader of HIF and/or inducing the expression of HIF and/or inducing the expression of at least one downstream effector of HIF in at least one organ and/or tissue and/or cell; wherein said method comprises the step of administering xenon or a pharmaceutical a composition comprising xenon to said organ and/or tissue and/or cell; and wherein said organ and/or tissue and/or cell is not any of brain, heart, embryonic nigral tissue, liver, lung, cornea, neurones, and endothelial cells of the intestine.

The present invention provides in another aspect the use of xenon as an HIF activator in the manufacture of an organ and/or tissue and/or cell protectant.

The present invention provides in further aspect the use of xenon as an HIF activator in the manufacture of an organ and/or tissue and/or cell protectant.

The present invention further provides the use of xenon as the sole organ and/or tissue and/or cell protectant in the manufacture of a pharmaceutical composition for the protection from injury of an organ and/or tissue and/or cell; wherein said pharmaceutical composition is administered before and/or after said organs and/or tissue and/or cell is cooled; preferably said organ and/or tissue and/or cell is cooled.

### Preferred Aspects

Preferred aspects are mentioned herein. Some preferred aspects of the present invention are now presented below.

Preferably, the organ and/or tissue and/or cell is one or more of: kidney, pancreas, reproductive organs, muscle, skin, fat, fertilised embryos and joints.

The term “organ” as used herein refers to a structure consisting of cells and tissues which is capable of performing at least one specific function.

The term “tissue” as used herein refers to an integrated collection of cells that performs at least one specific function.

Preferably the organ and/or tissue and/or cell is selected from the group consisting of: kidney; pancreas; reproductive organs; muscle; skin; fat; fertilised embryos; and joints—as organs or tissues thereof.

More preferably the organ or tissue is kidney or kidney tissue.

Preferably the organ and/or tissue and/or cell is an ex vivo organ and/or tissue and/or cell.

Preferably the organ and/or tissue and/or cell is an in vivo organ and/or tissue and/or cell.

Preferably xenon or a pharmaceutical composition comprising xenon is used as a sole organ and/or tissue and/or cell protectant.

Preferably the xenon or the pharmaceutical composition comprising xenon is administered to an organ and/or tissue and/or cell before said organ and/or tissue and/or cell is injured.

Preferably the xenon in the pharmaceutical composition is used in combination with a pharmaceutically acceptable carrier, diluent or excipient.

Preferably the xenon or pharmaceutical composition comprising xenon is administered to an organ and/or tissue and/or cell before said organ and/or tissue and/or cell is injured.

Preferably the xenon or pharmaceutical composition comprising xenon is administered to an organ and/or tissue and/or cell after said organ and/or tissue and/or cell is injured.

Preferably the xenon or pharmaceutical composition comprising xenon is administered to an organ and/or tissue and/or cell at the same time as said organ and/or tissue and/or cell is injured.

Preferably said invention further comprises one or more of:
(i) cooling said organ and/or tissue and/or cell;
(ii) perfusing and/or superfusing said organ and/or tissue and/or cell with one or more agents that supply energy to said organ and/or tissue and/or cell; and
(iii) perfusing and/or superfusing said organ and/or tissue and/or cell with one or more agents that decrease the energy requirements of said organ and/or tissue and/or cell;
   when said organ and/or tissue and/or cell is injured.

Preferably said invention further comprises one or more of:
(i) cooling said organ and/or tissue and/or cell;
(ii) supplying one or more blood nutrients from a source other than the normal blood and/or plasma supply; and
(iii) increasing the energy reserves of said organ and/or tissue and/or cell;
   before said organ and/or tissue and/or cell is injured.

Preferably said invention further comprises one or more of:
(i) administering at least one chelator (such as 2,2′-dipyridyl) and/or at least one converter of at least one reactive oxygen species;
(ii) administering at least one agent which decreases the levels of cytokines and/or chemokines;
(iii) cooling said organ and/or tissue and/or cell;
(iv) decreasing the energy requirements of said organ and/or tissue and/or cell;
(v) increasing the flow of urine from a subject (when said organ and/or tissue is an in vivo kidney);
(vi) performing dialysis (when said organ and/or tissue is an in vivo kidney);
   after said organ and/or tissue and/or cell is injured.

### Advantages

The present invention teaches that the vulnerability of an organ and/or tissue and/or cell (such as isolated organs and/or isolated tissues and/or isolated cells) to injury may be reduced by the administration of an HIF activator such as xenon. Without wishing to be bound by theory, the reduction in the extent of injury is effected through the modulation of effectors of processes that promote energy conservation and cell survival through increased oxygen delivery or facilitated metabolic adaptation to hypoxia. HIF activators may also exert other protective effects including: the development of neovascularisation for implantation of tissue constructs; protecting fertilised embryos which are implanted into a uterus; protection of the organ and/or tissue and/or cell from apoptosis; and improved wound healing.

Furthermore, the present invention is based on the surprising finding that only an HIF activator—such as xenon—needs to be administered to an organ and/or tissue and/or cell (such as isolated organs and/or isolated tissues and/or isolated cells) to act as an organ and/or tissue and/or cell protectant. In other words the HIF activator, such as xenon, is the sole organ and/or tissue and/or cell (such as isolated organs and/or isolated tissues and/or isolated cells) protectant.

Surprisingly the methods according to the present invention are more efficient at preventing and reducing the extent of injury to an organ and/or tissue and/or cell (such as isolated organs and/or isolated tissues and/or isolated cells) than the methods of the prior art—such as cooling below normal body temperature the organ prior to injury. Unexpectedly synergy is observed even when the HIF activator (such as xenon) and cooling are administered asynchronously to the tissue and/or organ such as brain.

### DETAILED DESCRIPTION

In one embodiment the organ and/or tissue and/or cell is an ex vivo organ and/or tissue and/or cell.

In an alternative embodiment the organ and/or tissue and/or cell is an in vivo organ and/or tissue and/or cell.

In one embodiment preferably said organ and/or tissue and/or cell is used for transplantation.

In an alternative embodiment preferably said organ and/or tissue and/or cell is not used for transplantation.

The term “transplant as used herein” refers to the transfer of an organ and/or tissue and/or cell from one part of a subject to another part of the same subject or to the transfer of an organ and/or tissue and/or cell from a subject to another subject.

In another embodiment preferably the organ and/or tissue and/or cell is used for implantation. Examples of implants include: muscle, skin, fat, fertilised embryos and joints.

The term “implantation” as used herein refers to the transfer of an organ and/or tissue and/or cell which has been cultured in vitro and/or prepared ex vivo before said organ and/or tissue and/or cell is transferred into a subject. Examples of such implants include the generation of fertilised embryos in vitro; the growth and culture of muscle and/or skin and/or pancreatic islets in vitro; the preparation of artificial joints ex vivo and the culturing of fat cells ex vivo—each of these may be then implanted into a subject.

The term “joint” as used herein refers to a joint which has been prepared ex vivo. The cells and/or tissue and/or cell are fashioned into a joint ex vivo on a biomaterial scaffold. This may be referred to as “tissue engineering”.

In one embodiment the organ and/or tissue and/or cell is not brain or heart. In a more preferred embodiment the organ and/or tissue and/or cell is not any of brain, heart, embryonic nigral tissue, liver, lung, cornea, neurones, and endothelial cells of the intestine.

The organ and/or tissue and/or cell may be one or more of: kidney, pancreas, lung, liver, reproductive organs, muscle, skin, fat, fertilised embryos, joints, and endothelium. Preferably the organ and/or tissue and/or cell is one or more of: kidney, pancreas, reproductive organs, muscle, skin, fat, fertilised embryos and joints. In a highly preferred embodiment the organ is kidney or a tissue thereof. Preferably, the tissue is not intestinal endothelium and/or parenchymal cells.

An example of a pancreatic tissue is pancreatic islets.

The HIF activator may be used as a protectant for isolated cells.

The term “isolated cell” as used herein refers to a cell which is removed from the tissue or organ in which it naturally occurs. Preferably isolated cells may be selected from one or more of the group consisting of: pancreatic cells, liver cells, fibroblast cells, bone marrow cells, myocytes, renal cells, endothelial cells, chondrocytes, osteocytes, and stem cells. Preferably isolated cells may be selected from one or more of the group consisting of: pancreatic cells, liver cells, fibroblast cells, bone marrow cells, myocytes, renal cells, endothelial cells (but not endothelial cells of the intestine), chondrocytes, osteocytes, and stem cells. Most preferably the isolated cells are renal cells.

Examples of tissues comprising endothelial cells are renal tubes and alveoli.

Preferably tissues for use in the present invention are renal tubes or alveoli. More preferably said tissue is a renal tube.

The organ and/or tissue and/or cell may be at any developmental stage—i.e. the organ and/or tissue and/or cell may be that of an adult, child, infant or foetus.

Organ and/or Tissue and/or Cell Protectant

The term “organ and/or tissue and/or cell protectant” as used herein refers to the ability of an HIF activator, such as xenon, to enable a vulnerable organ or tissue to withstand the injury that occurs when nutrients are withdrawn or when reactive oxygen species are provided or generated by an organ and/or tissue and/or cell—such injuries may occur during transplantations, implantations and surgery. Without wishing to be bound by theory, the mechanism by which an HIF activator, such as xenon, protects organs and/or tissues and/or cells is by inducing the expression of HIF and/or its downstream effectors—such as erythropoietin, vascular endothelial growth factor (VEGF), inducible nitric oxide synthetase (iNOS), glycolytic enzymes, bNIP3, PHD3, CAIX, and glucose transporter-1 as well as other genes that have a hypoxia responsive element in their promoter region. Alternatively or in addition, without wishing to be bound by theory, the mechanism by which an HIF activator, such as xenon, protects organs and/or tissues and/or cells is by reducing the degradation of HIF by reducing the expression of an upstream degrader such as PHD2.

Sole Organ and/or Tissue and/or Cell Protectant

In some embodiments of the present invention, the HIF activator—such as xenon—is used as a sole organ and/or tissue and/or cell protectant.

As used herein, the term “sole organ and/or tissue and/or cell protectant” refers to a pharmaceutical composition comprising an HIF activator (such as xenon) wherein said HIF activator is the only component which is at a dosage wherein it is capable of protecting an organ and/or tissue and/or cell from injury. In other words, no other agent (such as carbon monoxide) may be present in the pharmaceutical composition at a dosage wherein said agent is also capable of acting as an organ and/or tissue and/or cell protectant.

Accordingly, the HIF activator—such as xenon—may either be used in conjunction with another agent, compound or composition or element that does not exhibit organ and/or tissue and/or cell protectant properties or be used in conjunction with another agent, compound or composition or element that is present in an amount that does not exhibit organ and/or tissue and/or cell protectant properties.

An example of a composition wherein HIF activator acts as the sole organ and/or tissue and/or cell protectant is a gas comprising a mixture of xenon and oxygen. Another example is a gas comprising a mixture of xenon and ambient air.

Protection from Injury

The phrase “protection from injury” as used herein refers to the reduction in the extent of an injury to an organ and/or tissue and/or cell when compared to an organ and/or tissue and/or cell which has not been treated, in accordance with the present invention, with a pharmaceutical composition comprising an HIF activator.

Preferably the treated organ and/or tissue and/or cell has reduction of at least about 10%, more preferably at least about 15% in the extent of the injury when compared to an organ and/or tissue and/or cell which has not been treated with a pharmaceutical composition comprising an HIF activator. Said extent of injury may be determined by comparing the relevant function of an injured tissue and/or organ against that of a tissue and/or organ which has not been injured—for example, in the kidney the extent of injury may be determined by measuring the levels of creatinine in injured organs and uninjured organs; in the pancreatic islets the ability to control glycaemic may be measured in injured tissues and uninjured tissues. Alternatively or in addition, said extent of injury may be determined by comparing the histological score of an injured tissue and/or organ against that on a tissue and/or organ which has not been injured—for example, in general, the extent of cell necrosis may be measured; in the kidney the extent of tubular cell necrosis may be measured

As used herein, the term “injury” or “injured” refers to a reduction, when compared to the normal blood supply, and/or withdrawal of blood nutrients (such as oxygen and glucose and other energy substrates) supplied to an organ and/or tissue and/or cell; and/or a release of reactive oxygen species (such as hydrogen peroxide, hypocholrite, hydroxyl radicals, superoxide anions, and peroxynitrites) into an organ and/or tissue and/or cell. These injuries may result in cellular damage, apoptosis, and necrosis.

An organ and/or tissue and/or cell may be injured by one or more of the following: ischaemia; reperfusion; the application of clamps to a blood vessel(s) supplying an organ and/or tissue and/or cell; transplantation; implantation; hyperoxia; hyperthermia; trauma (both blunt and open); and sepsis. Ischaemia-reperfusion injury may occur in a variety of clinical settings, including reperfusion after thrombolytic therapy, coronary angioplasty, organ and/or tissue and/or cell transplantation, aortic cross-clamping or cardiopulmonary bypass. Reperfusion of ischaemic tissues results both in a local and systemic inflammatory response that, in turn, may result in widespread microvascular dysfunction and altered tissue barrier function. If severe enough, the inflammatory response after ischaemia-reperfusion may even result in the “systemic inflammatory response syndrome (SIRS)” or “multiple organ dysfunction syndrome (MODS)”, which account for up to 30-40% of intensive care unit mortality. Thus, ischaemia-reperfusion injury may extend beyond the ischaemic area at risk to include injury of remote, non-ischaemic organs.

### Hypoxia-Inducible Factor HIF and Activators Thereof

The term “HIF activator” as used herein refers to any element, compound or composition which is capable of inducing the synthesis of an HIF polypeptide (for example, through enhanced transcription of a nucleotide sequence encoding HIF, and/or enhanced stabilisation of the transcript, and/or enhanced translation). In addition, or alternatively, said HIF activator enhances the promoter activity at the hypoxia responsive elements of genes such as EPO and VEGF. A highly preferred example of an HIF activator is xenon.

Preferably the expression of HIF in an organ and/or tissue and/or cell treated with an HIF activator is increased by at least about 10%, preferably at least about 15%, more preferably at least about 20%, more preferably at least about 25% when compared to an organ and/or tissue and/or cell which has not been treated with an HIF activator.

The term “HIF-1α activator” as used herein refers to any element, compound or composition which is capable of inducing the synthesis of an HIF-1α polypeptide (for example, through enhanced transcription of a nucleotide sequence encoding HIF-1α, and/or enhanced stabilisation of the transcript, and/or enhanced translation). In addition, or alternatively, said HIF-1α activator enhances the promoter activity at the hypoxia responsive elements of genes such as EPO and VEGF. A highly preferred example of an HIF-1α activator is xenon.

The term “HIF-2α activator” as used herein refers to any element, compound or composition which is capable of inducing the synthesis of an HIF-2α polypeptide (for example, through enhanced transcription of a nucleotide sequence encoding HIF-2α, and/or enhanced stabilisation of the transcript, and/or enhanced translation). In addition, or alternatively, said HIF-2α activator enhances the promoter activity at the hypoxia responsive elements of genes such as EPO and VEGF. A highly preferred example of an HIF-2α activator is xenon.

Hypoxia-inducible factor 1 (HIF-1) is an oxygen-dependent transcriptional activator. HIF-1 consists of a constitutively expressed HIF-1 subunit and one of three subunits (HIF-1α, HIF-2α or HIF-3α) where the HIF-1α subunit is unique to HIF-1 (Lee et al 2004 Exp Mol Med 36:1-12; Semenza 2000 J Appl Physiol 88:1474-1480). HIF-1α is probably expressed in most tissues (see Semenza 2000 J Appl Physiol 88:1474-1480). HIF-2α has a more cell-type restricted (see Wiesener et al, FASEB J. 2003 February; 17(2):271-3).

The term “organ and/or tissue and/or cell that express HIF” as used herein refers to organs and/or tissues and/or cells (such as kidneys, blood vessels, pancreas, reproductive organs, muscles, skin, fat, fertilised embryos and joints) which express the polynucleotide sequence encoding the HIF polypeptide and, optionally, the HIF polypeptide.

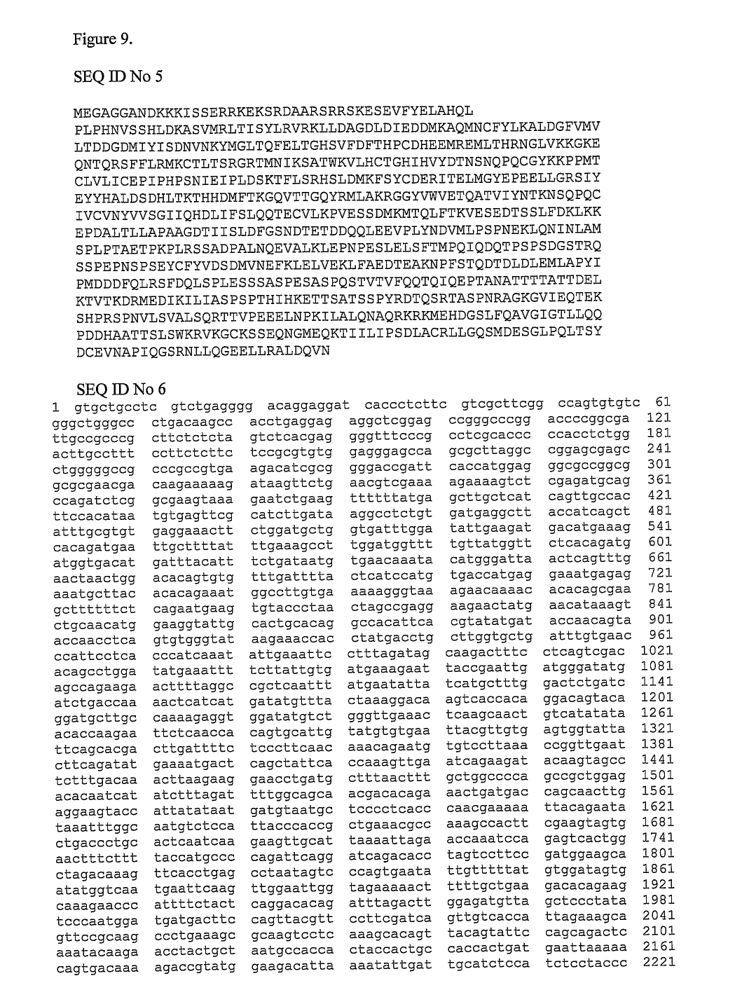

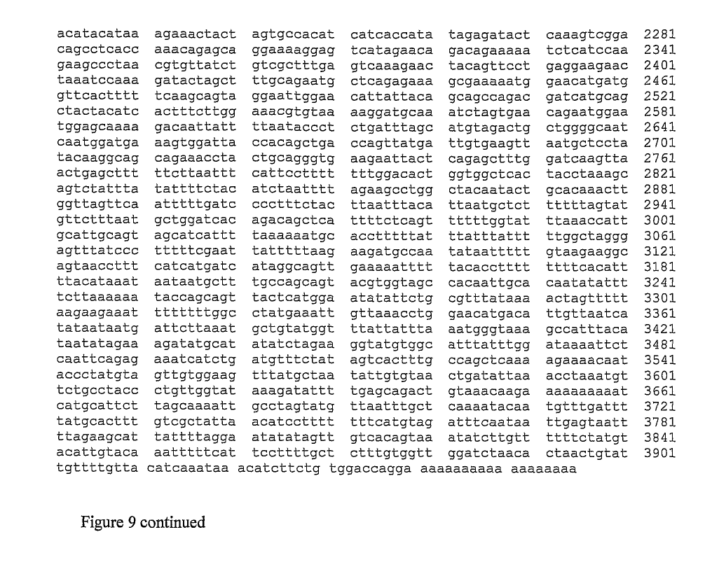

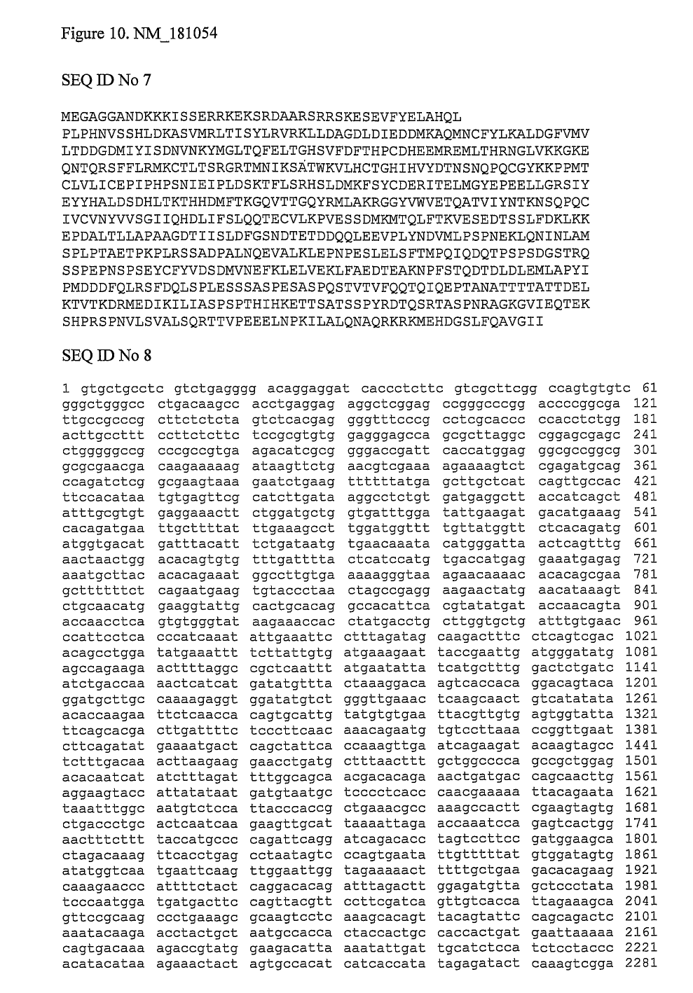

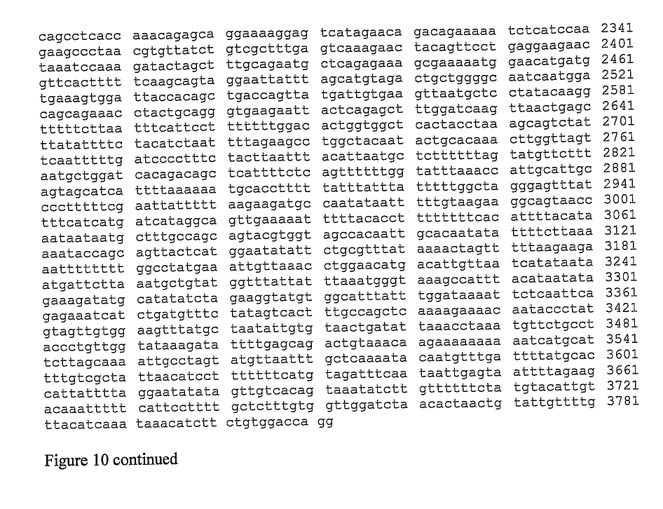

The polynucleotide sequence and the polypeptide sequence of HIF-1α are shown in FIGS. 9 and 10. These figures detail the sequences of accession numbers NM_{—}001530 and NM_{—}181054—both of which are Homo sapiens hypoxia-inducible factor 1, alpha subunit.

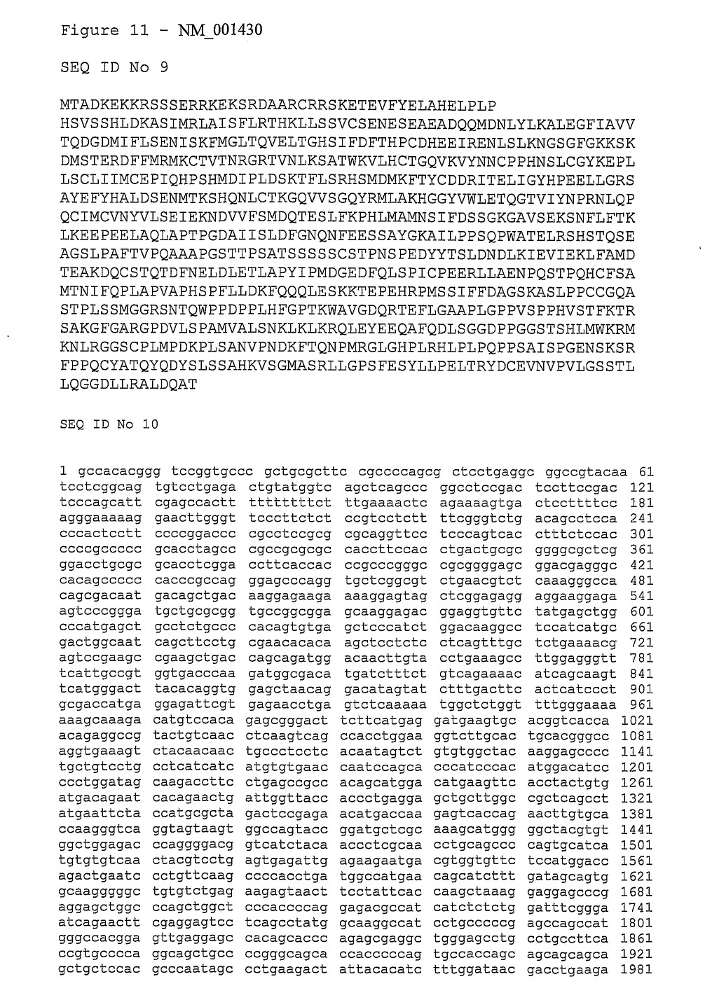

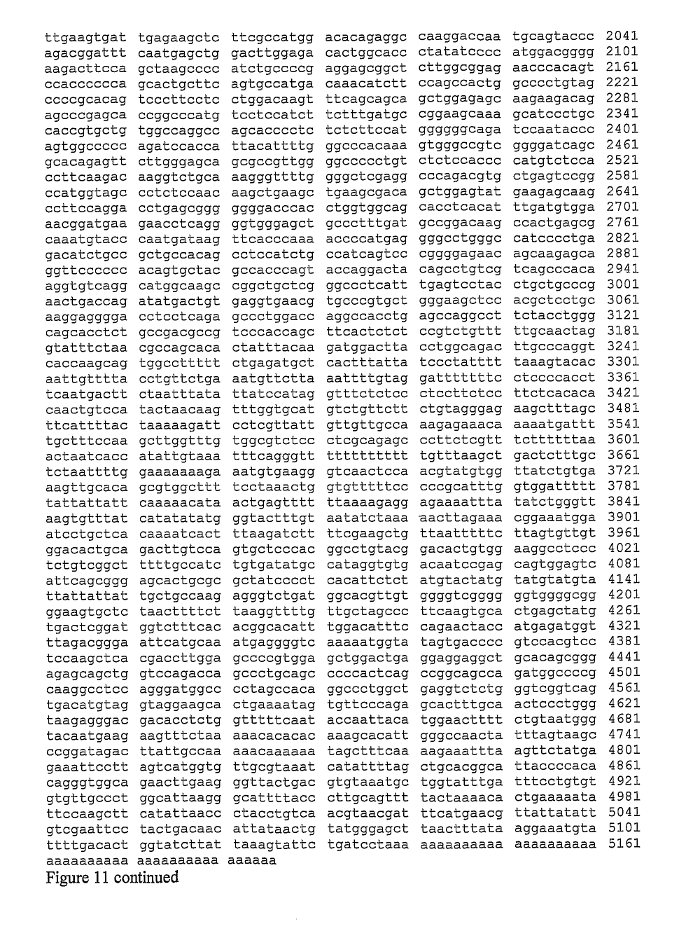

The polynucleotide sequence and the polypeptide sequence of HIF-2α are shown in FIGS. 11-13. These figures detail the sequences of accession numbers NM_{—}001430, BC051338 and U81984.1—each of which are Homo sapiens hypoxia-inducible factor 2, alpha subunit. HIF-2α is also referred to as Endothelial PAS domain protein 1 (EPAS-1), Member of PAS protein 2 (MOP2), Hypoxia-inducible factor 2 alpha, and HIF-1 alpha-like factor (HLF).

The expression of a gene encoding the polypeptide HIF and/or genes containing HIF responsive elements (HRE) in their promoter, enhancer or intronic regions can be detected in an organ and/or tissue and/or cell by the use of RT-PCR or even quantitative RT-PCR; these techniques are known in the art (see, for example, Sambrook et al (1989) Molecular cloning a laboratory manual, and Ausubel et al (1999) Short protocols in molecular biology) and kits such as the Qiagen QuantiTect Probe RT-PCR are available. The PCR amplification may be carried out using oligonucleotide primers derived from the gene encoding HIF-1α such as NM_{—}001530 and NM_{—}181054 and/or using oligonucleotide primers derived from the gene encoding HIF-2α such as NM_{—}001430 and BC051338. Furthermore, oligonucleotide primers derived from other HIF genes may be used. Hence an organ and/or tissue and/or cell can be evaluated to determine whether or not it expresses a nucleotide sequence encoding HIF polypeptide such as HIF-1α and/or HIF-2α. Also the organ and/or tissue and/or cell can be used to measure the activator effect of an actual or putative HIF activator.

The expression of the polypeptide HIF and/or polypeptides from genes containing HIF responsive elements (HRE) in their promoter, enhancer or intronic regions HIF in an organ and/or tissue and/or cell can be detected by the use of an antibody to HIF. Antibodies may be produced by standard techniques, such as by immunisation with the polypeptide of interest or by using a phage display library. The expression of polypeptide HIF-1α in an organ and/or tissue and/or cell can be detected by the use of an antibody to HIF-1α (such as monoclonal mouse anti-HIF-1α antibody (Novus Biologicals, UK)). The expression of polypeptide HIF-2α in an organ and/or tissue and/or cell can be detected by the use of an antibody to HIF-2α (such as polyclonal rabbit anti-HIF-2α (abcam)). These antibody can be used in immunohistochemical analysis of, for example, a tissue sample or for immunoblotting of proteins obtained from, for example, a tissue or an organ; these techniques are known in the art see, for example, Sambrook et al (1989) Molecular cloning a laboratory manual, Ausubel et al (1999) Short protocols in molecular biology, and Harlow and Lane (1988) Antibodies a laboratory manual). Hence an organ and/or tissue and/or cell can be evaluated to determine whether or not it expresses HIF (such as HIF-1α and/or HIF-2α). Also the organ and/or tissue and/or cell can be used to measure the activator effect of an actual or putative HIF activator.

In order to show that an agent is an HIF activator one or more of the following assays may be used:
a. RT-PCR to show an increase in the transcription of one or more HIF genes in an organ and/or tissue and/or cell when compared to an organ and/or tissue and/or cell which has not been treated with the agent;
b. immunoblotting and immunohistochemistry (for in situ demonstration) to show an increase in the expression of one or more HIF polypeptides in an organ and/or tissue and/or cell when compared to an organ and/or tissue and/or cell which has not been treated with the agent;
c. RT-PCR to show an increase in the transcription of one or more genes containing HIF responsive elements (HRE) in their promoter, enhancer or intronic regions in an organ and/or tissue and/or cell when compared to an organ and/or tissue and/or cell which has not been treated with the agent; and
d. immunoblotting and immunohistochemistry (for in situ demonstration) to show an increase in the expression of one or more polypeptides from genes containing HIF responsive elements (HRE) in their promoter, enhancer or intronic regions in an organ and/or tissue and/or cell when compared to an organ and/or tissue and/or cell which has not been treated with the agent.

The term “downstream effector of HIF” as used herein refers to a gene or polypeptide encoded by said gene whose expression is induced by the expression of the nucleotide sequence encoding HIF and/or the HIF polypeptide—in other words, HIF responsive genes.

Examples of downstream effectors of HIF are: erythropoietin, VEGF, iNOS, glycolytic enzymes, bNIP3, PHD3, CAIX, and glucose transporter-1 both the polypeptides and the nucleotide sequences encoding said polypeptides as well as other genes that have a hypoxia responsive element in their promoter region. HIF responsive genes include genes with functions in cellular energy metabolism, iron metabolism, catecholamine metabolism, vasomotor control and angiogenesis (Ratcliffe et al J Exp Biol. 1998 April; 201(Pt 8):1153-62; Wiesener and Maxwell 2003 Ann Med 35:183-190).

In a preferred aspect the downstream effector is selected from the group consisting of: erythropoietin, vascularendothelial growth factor (VEGF), inducible nitric oxide synthetase (iNOS), glycolytic enzymes, NIP3, prolyl hyroxylase 3 (PHD3), CAIX, glucose transporter-1, transferrin, transferrin receptor, ceruloplasmin, glucose transporter-3, hexokinase 1, hexokinase 2, LDH-A, PGK 1, aldolase A, aldolase C, phosphofructokinase L, pyruvate kinase M, enolase 1, triose phosphate isomerase, p21, NIX, insulin-like growth factor 2, IGFBP 1, IGFBP 2, IGFBP 3, VEGF-receptor FLT-1, plasminogen activator inhibitor 1, TGFβ3, endoglin, nitric oxide synthase 2, endothelin 1, a1B-adrenoceptor, adrenomedullin, heme oxygenase 1, carbonic anhydrase 9, adenylate kinase 3, prolyl-4-hydroxylase a1, p35srj, intestinal trefoil factor, leptin. More preferably the downstream effector is selected from the group consisting of: erythropoietin, vascularendothelial growth factor (VEGF), inducible nitric oxide synthetase (iNOS), glycolytic enzymes, NIP3, PHD3, CAIX, and glucose transporter-1. In a more preferred embodiment the downstream effector is erythropoietin (EPO). In a highly preferred embodiment the downstream effector is erythropoietin (EPO).

One example of an upstream degrader of HIF is prolyl hyroxylase 2 (PHD2).

The term “upstream degrader of HIF” as used herein refers to a gene or polypeptide encoded by said gene whose expression reduces the expression of the nucleotide sequence encoding HIF and/or the amounts of HIF polypeptide. Thus a reduction in the expression of such an upstream degrader of HIF will result in an increase in the expression of the nucleotide sequence encoding HIF and/or the HIF polypeptide.

A modulation (such as a reduction or induction) in the expression of a gene or polypeptide encoded by said gene is measured by comparing the levels in an organ and/or tissue and/or cell treated with a HIF activator (such as xenon) with suitable controls which have not been treated with a HIF activator.

### Administration of HIF Activator to a Subject

The term “HIF activator” includes a single type of activator or a mixture of HIF activators—wherein each of which is capable of exhibiting organ and/or tissue and/or cell protectant properties. In some preferred aspects, just one type of HIF activator is used. Preferably the HIF activator is, or includes, xenon. More preferably, the HIF activator is just xenon.

The HIF activator composition can be applied to a subject by various techniques; these techniques will be chosen depending on the particular use and the type of HIF activator composition. Typically, the pharmaceutical compositions for use as described herein may be administered by one or more of the following methods: intravascular administration (either by bolus administration or infusion), transdermal administration, inhalation, perfusion, superfusion, washing, submersion and topical application. Preferably said administration is by one or more of the following: inhalation, perfusion, and superfusion. Said administration will ensure a sufficient concentration of the HIF activator, such as xenon, in the blood and/or plasma.

The term “perfusion” as used herein refers to the passage of a liquid through the blood vessels of an organ and/or tissue and/or cell.

The term “superfusion” as used herein refers to maintaining the metabolic or physiological activity of an isolated organ and/or tissue and/or cell by providing a continuous flow of a sustaining medium. Examples of isolated organ and/or tissue and/or cell include tissue engineered implants and fertilised embryos.

In one aspect, the HIF activator composition is administered to a subject to the extent that there is a sufficient concentration of the HIF activator, such as xenon, in the blood and/or plasma of the organ and/or tissue and/or cell.

In one embodiment the HIF activator composition may be administered as a gas. Preferably the HIF activator may be admixed with another gas, such as oxygen.

In another embodiment the HIF activator is admixed with ambient air instead of oxygen.

Preferably the HIF activator is used as the sole organ and/or tissue and/or cell protectant. When the HIF activator is used as the sole organ and/or tissue and/or cell protectant then no other agent (such as carbon monoxide) may be added at a dosage wherein said agent is capable of acting as an organ and/or tissue and/or cell protectant; preferably said other agent is not capable of acting as an organ and/or tissue and/or cell protectant at any dosage.

Compressed or pressurised gas for use in the present invention can be obtained from any commercial source, and in any type of vessel appropriate for storing compressed gas. For example, compressed or pressurised gases can be obtained from any source that supplies compressed gases, such as xenon, oxygen etc. for medical use. The pressurised gases can be provided such that all gases of the desired final composition are mixed in the same vessel. Optionally, the present invention can be performed by using multiple vessels containing individual gases.

Alternatively, the HIF activator, such as xenon, may be administered to an organ and/or tissue and/or cell as an HIF activator-saturated solution (such as a xenon-saturated solution).

One example of how an HIF activator composition, such as xenon, may be administered is by the use of an inhalation apparatus which is already used for anaesthesia by inhalation. If a cardiopulmonary bypass machine or another artificial breathing apparatus is used then the HIF activator, such as xenon, can be added directly in the machine and requires no further apparatus. On an ambulant basis, e.g., in case of an emergency, it is even possible to use simpler inhalators, which mix the HIF activator such as xenon with the ambient air during the process of inhalation. In this connection, it is also possible to adapt the HIF activator, such as xenon, concentration and the timing of the HIF activator, such as xenon, application in a simple manner to the therapeutic requirements. For example, it might be advantageous to use mixtures of xenon with other gases harmless to humans, e.g., oxygen, nitrogen, ambient air etc.

In another example, donor organs and/or tissues and/or cells may be treated by the donor inhaling the HIF activator composition prior to harvesting of the donor organ and/or tissue and/or cell. Alternatively, or in addition, the donated organ and/or tissue may be treated ex vivo by superfusion or perfusion with the HIF activator composition immediately prior to implantation. Tissues and/or organs for implantation may be treated by perfusion with the HIF activator composition prior to implantation

In one aspect of the present invention the HIF activator composition is administered to a subject by inhalation. Said inhalation results in a sufficient concentration of the HIF activator, such as xenon, in the blood and/or plasma.

Preferably the HIF activator composition comprises at least about 70%, preferably about 75%, more preferably about 80%, most preferably about 90% of the HIF activator.

In another embodiment, the HIF activator composition comprises an HIF activator:oxygen mixture of about 70:30%, preferably about 75:25% by volume, more preferably about 80:20% by volume, most preferably about 90:10% by volume.

Preferably the HIF activator composition is administered to an organ and/or tissue and/or cell for up to about 2 hours, preferably up to about 3 hours, preferably for up to about 4 hours, preferably for up to about 8 hours, preferably for up to about 12 hours, more preferably for up to about 16 hours, more preferably for up to about 20 hours and more preferably up to about 24 hours.

Preferably the HIF activator composition is administered to an organ and/or tissue and/or cell up to about 2 hours prior to injury, preferably up to about 3 hours, preferably for up to about 4 hours, preferably for up to about 8 hours, preferably for up to about 12 hours, more preferably for up to about 16 hours, more preferably for up to about 20 hours and more preferably up to about 24 hours.

The HIF activator may be administered in combination with a pharmaceutically acceptable carrier, diluent or excipient. By way of example, in the pharmaceutical compositions of the present invention, the HIF activator may be admixed with any suitable binder(s), lubricant(s), suspending agent(s), coating agent(s), solubilising agent(s) selected with regard to the intended route of administration and standard pharmaceutical practice. Nevertheless when the HIF activator (such as xenon) is used as the sole organ and/or tissue and/or cell protectant then no other pharmaceutically acceptable carrier, diluent or excipient may be added at a dosage wherein said pharmaceutically acceptable carrier, diluent or excipient is capable of acting as an organ and/or tissue and/or cell protectant. Preferably said pharmaceutically acceptable carrier, diluent or excipient is not capable of acting as an organ and/or tissue and/or cell protectant at any dosage.

The HIF activator may be administered in combination with one or more different HIF activators.

The HIF activator, such as xenon, may be administered in combination with a compound or agent that has pharmaceutical properties (but if the HIF activator is the sole organ and/or tissue and/or cell protectant then these properties are not organ and/or tissue and/or cell protectant properties). An example of a pharmaceutical property is an anaesthetic. An example of an anaesthetic is sevoflurane. However said when the HIF activator is used as the sole organ and/or tissue and/or cell protectant then said anaesthetic is not present in a dosage wherein said anaesthetic is capable of acting as an organ and/or tissue and/or cell protectant. Preferably said anaesthetic is not capable of acting as an organ and/or tissue and/or cell protectant at any dosage.

The composition comprising the HIF activator, such as xenon, as described herein may comprise one or more of the following agents: sevoflurane, isoflurane, desflurane, and dexmedetomidine. Nevertheless when an HIF activator, such as xenon, is used as the sole organ and/or tissue and/or cell protectant then no agent which is capable of acting as an organ and/or tissue and/or cell protectant may be added to the composition at a dosage wherein said agent is capable of acting as an organ and/or tissue and/or cell protectant. Preferably said agent is not capable of acting as an organ and/or tissue and/or cell protectant at any dosage.

The pharmaceutical compositions comprising an HIF activator, such as xenon, as described herein may be for human administration or animal administration.

The concentration of an HIF activator, such as xenon, employed in a pharmaceutical composition may be the minimum concentration required to achieve the desired clinical effect. It is usual for a physician to determine the actual dosage that will be most suitable for an individual patient, and this dose will vary with the age, weight and response of the particular patient. There can, of course, be individual instances where higher or lower dosage ranges are merited.

The pharmaceutical composition comprising an HIF activator, such as xenon, as described herein may also be used as an animal medicament. Such an animal medicament (or veterinary composition) comprises an HIF activator, such as xenon, and a veterinarily acceptable diluent, excipient or carrier.

For veterinary use, the veterinarily acceptable composition described herein is typically administered in accordance with normal veterinary practice and the veterinary surgeon will determine the dosing regimen and route of administration which will be most appropriate for a particular animal.

In one embodiment the composition comprising the HIF activator, such as xenon, as described herein is administered to an organ and/or tissue and/or cell before the organ and/or tissue and/or cell is injured.

In another embodiment the composition comprising the HIF activator, such as xenon, as described herein is administered to an organ and/or tissue and/or cell after the organ and/or tissue and/or cell is injured.

In a further embodiment the composition comprising the HIF activator, such as xenon, as described herein is administered to an organ and/or tissue and/or cell at the same time as the organ and/or tissue and/or cell is injured.

In another embodiment the composition comprising the HIF activator, such as xenon, as described herein is administered to an organ and/or tissue and/or cell before and after the organ and/or tissue and/or cell is injured.

In another embodiment the composition comprising the HIF activator, such as xenon, as described herein is administered to an organ and/or tissue and/or cell before the organ and/or tissue and/or cell is injured and during injury to the organ and/or tissue and/or cell.

In another embodiment the composition comprising the HIF activator, such as xenon, as described herein is administered to an organ and/or tissue and/or cell before, during and after the organ and/or tissue and/or cell is injured.

The composition comprising the HIF activator, such as xenon, as described herein may comprise one or more of the following agents: sevoflurane, isoflurane, desflurane, and dexmedetomidine Nevertheless when an HIF activator, such as xenon, is used as the sole organ and/or tissue and/or cell protectant then no agent which is capable of acting as an organ and/or tissue and/or cell protectant may be added to the composition at a dosage wherein said agent is capable of acting as an organ and/or tissue and/or cell protectant. Preferably said agent is not capable of acting as an organ and/or tissue and/or cell protectant at any dosage.

A pharmaceutical composition comprising an HIF activator may be administered to a subject before and/or after and/or during injury to an organ and/or tissue and/or cell. In addition, said subject may also receive one or more of the following treatments prior to the injury:
(i) avoiding administering agents which injure organs and/or tissue and/or cell (such as aminoglycosides for the kidney, acetaminophen and alcohol for the liver, daunorubicin for the lung);
(ii) cooling said organ and/or tissue and/or cell to a temperature below normal body temperature;
(iii) evacuating the intraluminal contents when said organ and/or tissue and/or cell thereof is the intestine;
(iv) supplying one or more blood nutrients from a source other than the normal blood supply to said organ and/or tissue and/or cell;
(v) increasing the energy reserves of said organ and/or tissue and/or cell compared to the normal levels of energy reserves;
(vi) ischaemic preconditioning; and
(vii) hypoxic preconditioning.

In addition or alternatively, one or more of the following procedures may be carried out after the organ and/or tissue and/or cell has been injured:
(i) treating the organ and/or tissue and/or cell with at least one chelator and/or at least one converter of at least one reactive oxygen species;
(ii) administering at least one agent which decreases the levels of cytokines and/or chemokines in the organ and/or tissue and/or cell;
(iii) cooling said organ and/or tissue and/or cell below normal body temperature;
(iv) decreasing the energy requirements of the organ and/or tissue and/or cell;
(v) increasing the flow of urine from a subject when the organ is an in vivo kidney; and
(vi) performing dialysis (such as peritoneal and/or haemeodialysis) on the subject when the organ is an in vivo kidney.

A pharmaceutical composition comprising an HIF activator may be administered to an ex vivo organ and/or tissue and/or cell before and/or after and/or during injury to said organ and/or tissue and/or cell. In addition said organ and/or tissue and/or cell may undergo one or more of the following procedures before and/or after and/or during injury to said organ and/or tissue and/or cell:
(i) cooling said organ and/or tissue and/or cell to below normal body temperature;
(ii) perfusing and/or superfusing said organ and/or tissue and/or cell with one or more agents that supply energy to said organ and/or tissue and/or cell; and
(iii) perfusing and/or superfusing said organ and/or tissue and/or cell with one or more agents that decrease the energy requirements of said organ and/or tissue and/or cell.

Preferably the method of reducing the expression of at least one upstream degrader of HIF and/or inducing the expression of HIF and/or inducing the expression of at least one downstream effector of HIF in at least one organ and/or tissue and/or cell—wherein said method comprises the step of administering a composition comprising an HIF activator, such as xenon, to said organ and/or tissue and/or cell—further comprises one or more of the following steps:
(i) cooling said organ and/or tissue and/or cell;
(ii) perfusing and/or superfusing said organ and/or tissue and/or cell with one or more agents that supply energy to said organ and/or tissue and/or cell; and
(iii) perfusing and/or superfusing said organ and/or tissue and/or cell with one or more agents that decrease the energy requirements of said organ and/or tissue and/or cell;
   when said organ and/or tissue and/or cell is injured.

Preferably the method of reducing the expression of at least one upstream degrader of HIF and/or inducing the expression of HIF and/or inducing the expression of at least one downstream effector of HIF in at least one organ and/or tissue and/or cell—wherein said method comprises the step of administering a composition comprising an HIF activator, such as xenon, to said organ and/or tissue and/or cell—further comprises one or more of the following steps:
(i) cooling said organ and/or tissue and/or cell;
(ii) supplying one or more blood nutrients from a source other than the normal blood and/or plasma supply;
(iii) increasing the energy reserves of said organ and/or tissue and/or cell;
(vi) ischaemic preconditioning; and
(vii) hypoxic preconditioning
   before said organ and/or tissue and/or cell is injured.

Preferably the method of reducing the expression of at least one upstream degrader of HIF and/or inducing the expression of HIF and/or inducing the expression of at least one downstream effector of HIF in at least one organ and/or tissue and/or cell—wherein said method comprises the step of administering a composition comprising an HIF activator, such as xenon, to said organ and/or tissue and/or cell—further comprises one or more of the following steps:
(i) administering at least one chelator and/or at least one converter of at least one reactive oxygen species;
(ii) administering at least one agent which decreases the levels of cytokines and/or chemokines;
(iii) cooling said organ and/or tissue and/or cell;
(iv) decreasing the energy requirements of said organ and/or tissue and/or cell;
(v) increasing the flow of urine from a subject (when said organ and/or tissue is an in vivo kidney);
(vi) performing dialysis (when said organ and/or tissue is an in vivo kidney); after said organ and/or tissue and/or cell is injured.

The term “cooling said organ and/or tissue and/or cell” as used herein refers to cooling the organ and/or tissue and/or cell to a temperature below normal body temperature. Cooling may be applied either locally or generally. Such cooling may be carried out by perfusing and/or superfusing an in vivo or ex vivo organ and/or tissue and/or cell with at least one liquid which is at a temperature below normal body temperature. Alternatively an ex vivo organ and/or tissue and/or cell may be submerged in at least one liquid which is at a temperature below normal body temperature.

Therapeutic cooling is reviewed in Tisherman et al (1999) (Surg Clin North Am. 79(6):1269-89).

Suitable temperatures include cooling an organ or tissue to about 35° C., about 30° C., about 25° C., about 20° C., about 15° C., about 10° C., or about 4° C. when said organ and/or tissue and/or cell is an ex vivo organ and/or tissue and/or cell.

Suitable temperatures include cooling an organ or tissue to about 35° C., about 30° C., about 25° C., about 20° C., about 15° C., about 10° C., or about 4° C. when said organ and/or tissue and/or cell is an in vivo organ and/or tissue and/or cell.

The term “evacuating the intraluminal contents” as used herein refers to the removal of the contents within an intestine. Such removal may be carried out by irrigating or flushing said intestine with a solution that may be saline or an antibiotic containing solution.

The term “agents that supply energy to said organ and/or tissue and/or cell” as used herein refers to agents which are capable of providing an organ and/or tissue and/or cell with a source of energy. Examples of such agents include glucose, insulin, and potassium solution. In other words any solution that is capable of increasing the production of ATP.

The term “decreasing the energy requirements of said organ and/or tissue and/or cell” as used herein refers to an agent which is capable of decreasing the energy reserves of said organ and/or tissue and/or cell when compared to an organ and/or tissue and/or cell which has not been treated with said agent. One example of such an agent is a cardioplegia solution. A cardioplegia solution is a solution which comprises high levels of potassium and magnesium. Without wishing to be bound by theory, a cardioplegia solution is capable of decreasing the energy requirements of an organ and/or tissue and/or cell by reducing the likelihood of membrane depolarisation. By decreasing the occurrence of membrane depolarisation the energy requirements of an organ and/or tissue and/or cell is decreased. Said agent may be supplied by perfusing and/or superfusing an in vivo or ex vivo organ and/or tissue and/or cell with the agent. Alternatively an ex vivo organ and/or tissue and/or cell may be submerged in the agent.

As used herein the term “supplying one or more blood nutrients from a source other than the normal blood and/or plasma supply” refers to: transfusion blood and/or plasma (said blood and/or plasma is either obtained from the subject on a prior occasion or is obtained from another blood compatible subject); or a sterile aqueous solution which comprises enough salts or monosaccharides to make the solution isotonic with blood and comprises blood nutrients (such as glucose, proteins, peptides, lipids, fatty acids, and cholesterol); or blood plasma. Said blood nutrients may be supplied by perfusing and/or superfusing an in vivo or ex vivo organ and/or tissue and/or cell with the above-mentioned sources of blood nutrients. Alternatively an ex vivo organ and/or tissue and/or cell may be submerged in the above-mentioned blood nutrients.

As used herein the term “increasing the energy reserves of said organ and/or tissue and/or cell” refers to at least one agent which is capable of increasing the energy reserves of said organ and/or tissue and/or cell being administered to said organ and/or tissue and/or cell such that when the treated organ and/or tissue and/or cell is compared to an organ and/or tissue and/or cell which has not been treated with said agent then the energy reserves have been increased. Examples of such agents include glucose, insulin, and potassium solution. In other words any solution that is capable of increasing the production of ATP. Said agent may be supplied by perfusing and/or superfusing an in vivo or ex vivo organ and/or tissue and/or cell with the agent. Alternatively an ex vivo organ and/or tissue and/or cell may be submerged in the agent.

A chelator may be supplied by perfusing and/or superfusing an in vivo or ex vivo organ and/or tissue and/or cell with the chelator. Alternatively an ex vivo organ and/or tissue and/or cell may be submerged in a chelator. The term “chelator” is used in its normal sense in the art—i.e. an agent which is capable of combining with free metal ions. Examples of chelators include iron chelators and transition metal ion chelators. Examples of chelators include 2,2′-dipyridyl and desferrioxamine.

As used herein the term “converter of at least one a reactive oxygen species” refers to an agent which is capable of converting at least one reactive oxygen species to non-reactive oxygen species. Said agent may be supplied by perfusing and/or superfusing an in vivo or ex vivo organ and/or tissue and/or cell with the agent. Alternatively an ex vivo organ and/or tissue and/or cell may be submerged in the agent.

The term “administering at least one agent which decreases the levels of cytokines and/or chemokines” as used herein refers to the use of an agent which is capable of decreasing the energy reserves of said organ and/or tissue and/or cell when compared to a organ and/or tissue and/or cell which has not been treated with said agent. Examples of such agents include lipoxins. Said agent may be supplied by perfusing and/or superfusing an in vivo or ex vivo organ and/or tissue and/or cell with the agent. Alternatively an ex vivo organ and/or tissue and/or cell may be submerged in the agent.

As used herein the term “the flow of urine from a subject is increased” refers to an increase in the amount of urine which is excreted from a subject. Such an increase may be achieved by increasing the intake of a composition comprising water by a subject and/or by intravascular administration of a composition comprising water.

### Xenon

In a preferred embodiment the HIF activator as described herein is xenon.

Preferably the HIF activator composition comprises xenon or is xenon.

The term “xenon” as used herein is not intended to restrict the present invention to a gas or liquid of pure xenon. The term also encompasses a composition comprising xenon—such as a mixture of xenon and oxygen. Nevertheless, when xenon is used as the sole organ and/or tissue and/or cell protectant then no agent (such as carbon monoxide) may be added to a mixture at a dosage wherein said agent is capable of acting as an organ and/or tissue and/or cell protectant. Preferably said agent is not capable of acting as an organ and/or tissue and/or cell protectant at any dosage.

Xenon (Xe) is an atom (atomic number 54) existing in the ambient atmosphere in low concentration (0.0000086% or 0.086 part per million (ppm) or 86 parts per million (ppb)). When purified it is presented as a gas in normobaric situations. Xenon is one of the inert or “Nobel” gases including also argon and Krypton. Due to its physiochemical properties xenon gas is heavier then normal air, with a specific gravity or density of 5.887 g/l and its oil/gas partition coefficient is 1.9 and “blood/gas” partition coefficient of about 0.14.

In concentrations of 10-70 vol. % in combination with oxygen, xenon exhibits anaesthetic effects. A number of studies in humans have looked at the effects of both hyperbaric and normobaric effects of xenon and shown dose dependent analgesic properties similar to those of nitrous oxide and that xenon in higher concentration exhibits anaesthetic properties and creates a drug induced stage of sleep and depression of response to painful stimuli (EP-A-0 864 328; EP-A-0 864 329).

The uptake of xenon via the respiratory system and the transport into the brain are already known from the use of xenon as an anaesthetic agent. It can also be assumed, from its use as anaesthetic agent, that the use of xenon has no damaging effect on an organism. Moreover, studies have shown that xenon exposure does not induce significant toxic effects on main organs (Natale at al 1998; Applied Cardiopulmonary Pathophysiology, 7:227-233).

Helium may be added to xenon gas since helium is a molecule of small size it may function as carrier for the more voluminous xenon. Furthermore, further gases having medical effects may be added to the xenon composition, e.g. NO or CO₂. In addition, depending on the disease to be treated other medicaments which are preferably inhalable may be added, e.g. cortisons, antibiotics etc. However when xenon is used as the sole organ and/or tissue and/or cell protectant then no other agent (such as carbon monoxide) may be added at a dosage wherein said agent is capable of acting as an organ and/or tissue and/or cell protectant. Preferably said agent is not capable of acting as an organ and/or tissue and/or cell protectant at any dosage.

Xenon can be administered to an organ and/or tissue and/or cell as a xenon-saturated solution. One way in which a xenon-saturated solution may be prepared is to expose a buffered physiologic salt solution to 100% xenon, or alternatively 80% xenon/20% oxygen, in an air-tight plastic bag and mix for one hour on a shaker. The gas atmosphere is changed at least once and the mixing procedure repeated. Then a complete saturation of the buffer with the gas (mixture) is achieved (Wilhelm S, Ma D, Maze M, Franks N P (2002) Effects of xenon on in vitro and in vivo models of neuronal injury Anesthesiology. 96:1485-91).

A xenon-saturated solution is particularly useful for transplantation and implantation purposes. If the organ and/or tissue and/or cell is maintained during transport or during the pre-operation phase in such a solution, a considerable reduction of the rate of apoptosis in the organ and/or tissue and/or cell can be observed.

### Cancer Treatment

Radiotherapy and/or chemotherapy causes injury to cancerous cells and/or tissue and/or cells and/or organs and healthy cells and/or tissue and/or cells and/or organs.

Preferably the organ and/or tissue and/or cell is a cancerous and/or pre-cancerous organ and/or tissue and/or cell.

In one aspect the present invention provide the use of xenon in the manufacture of a pharmaceutical composition for the treatment of at least one cancerous and for pre-cancerous organ and/or tissue and/or cell; wherein said xenon is used in conjunction with (i.e. sequentially or simultaneously) with at least one vector comprising an HIF responsive element.

Thus, in one aspect the present invention provides the use of xenon in the manufacture of a pharmaceutical composition for the treatment of at least one cancerous and/or pre-cancerous organ and/or tissue and/or cell; wherein said organ and/or tissue and/or cell comprises or has been exposed to at least one vector comprising an HIF responsive element.

HIF responsive elements are known in the art (see Wiesener M S and Maxwell P H (2003): HIF and oxygen sensing: As important to life as the air we breathe. Ann of Medicine 35:183).

The vector may any suitable vector capable of delivering the HIF responsive element to the organ or tissue. The vector may be a viral vector—such as a retroviral vector. In addition or in the alternative the vector(s) may be a non-viral vector, such as a chemical vector—such as a liposome.

Preferably said vector comprises a polynucleotide sequence capable of expressing a suicide gene wherein said polynucleotide sequence is operably linked to an HIF responsive element.

The term “suicide gene” as used herein refers to a gene which, when expressed, causes cell necrosis and/or cell apoptosis.

Vectors comprising suicide gene operably linked to an HIF responsive element are mentioned in Scott and Greco (Cancer Metastasis Rev. 2004 August-December;23(3-4):269-76) and Ruan and Dean (Curr Opin Investig Drugs. 2001 June; 2(6):839-43).

### Additional Treatments

A subject may receive one or more of the following treatments before and/or after and/or during injury to an organ and/or tissue and/or cell:
(i) radiation therapy;
(ii) chemotherapy;
(iii) cryotherapy;
(iv) hyperthermia;
(v) hypoxia; and
(vi) nutritional supplementation.

Thus, the present invention (such as the use or method as described herein) may further comprise one or more of the above-mentioned treatments.

In these embodiments the organ and/or tissue and/or cell may be cancerous and/or pre-cancerous.

In these embodiments preferably the organ and/or tissue and/or cell is kidney.

### EXAMPLES

The present invention is further described by way of examples and with reference to the following figures.

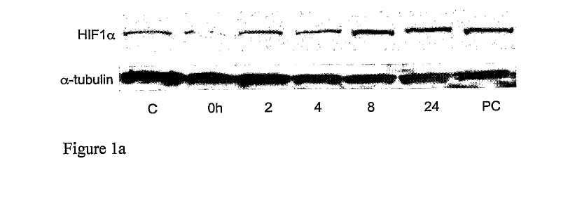

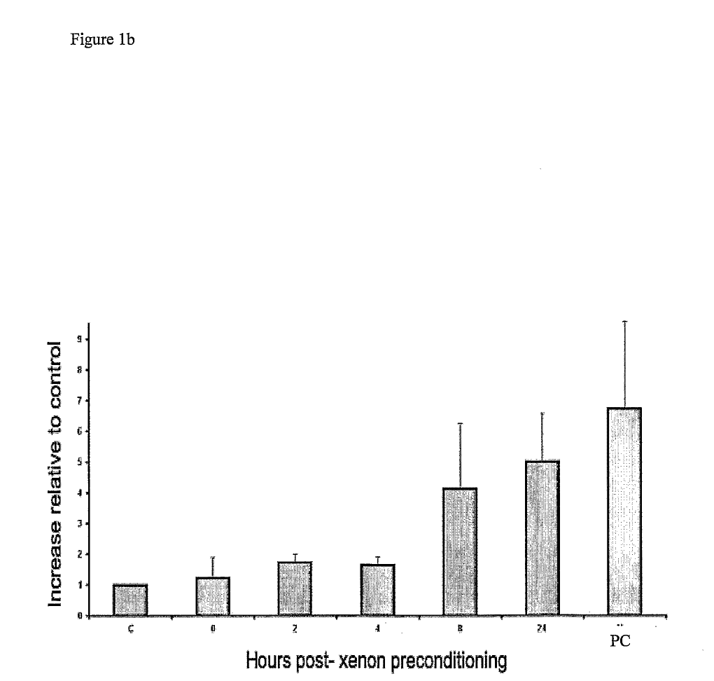

FIG. 1 shows the changes over time in the levels of the polypeptides HIF-1α, and the control α-tubulin, in the kidney of adult C57B6 mice exposed for 2 hour to 75% xenon. C=naïve control; 0-24 hr=the time point at which tissues were harvested after exposure to 75% xenon for 2 hours; PC=positive control (wherein said animal was exposed to 8% O₂ for 1 hr).

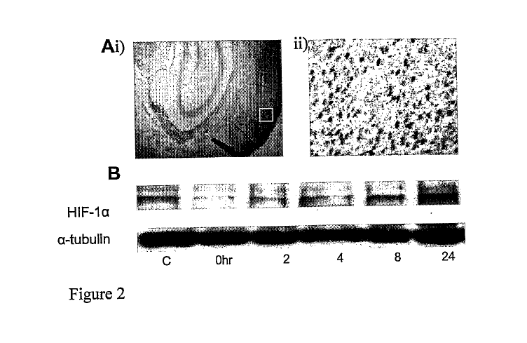

FIG. 2 shows the expression of the polypeptide HIF-1α, and the control polypeptide α-tubulin, in the brains of rats which were exposed to 75% xenon for 2 hours. Said brains were assessed by immunohistochemistry (A) and western blotting (B). A: HIF-1α positive cells were clearly detected in cortex 6 hrs after exposure for 2 hours to 75% xenon. B: The changes in the expression of HIF-1α, and the control α-tubulin, over time in neonatal rat brain exposed for 2 hours to 75% xenon. C=naïve control; 0-24 hr=time point at which tissues were harvested after 75% xenon exposure for 2 hours.

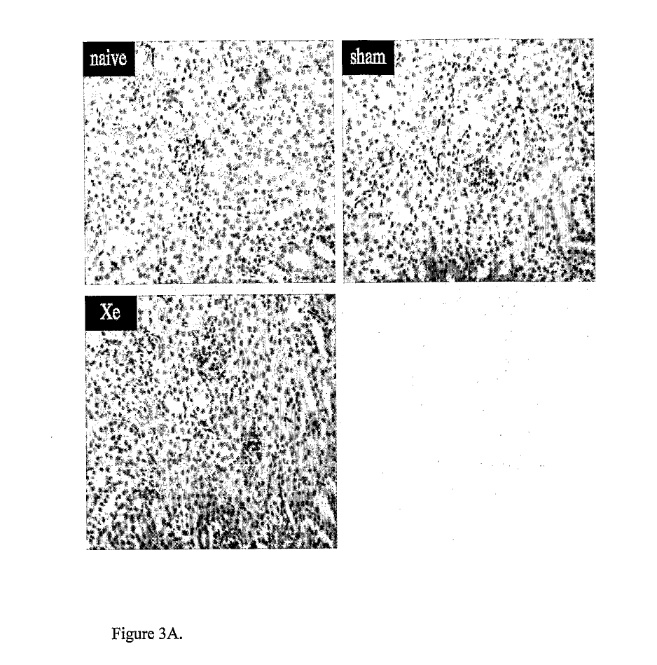

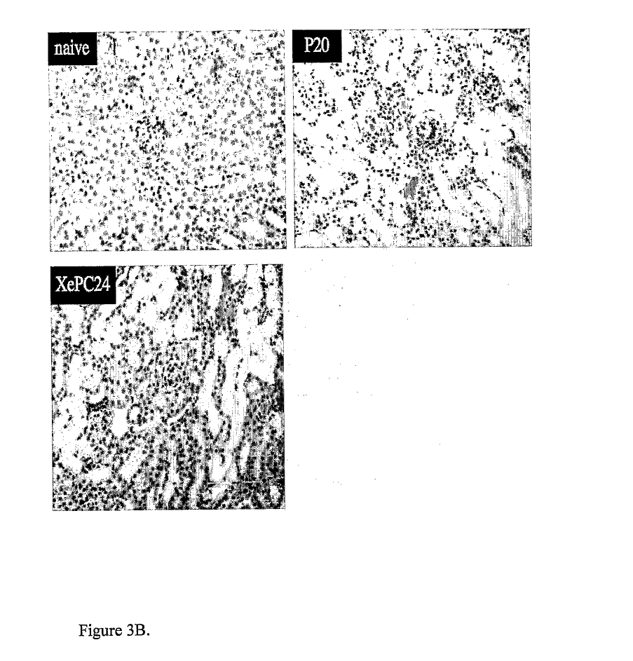

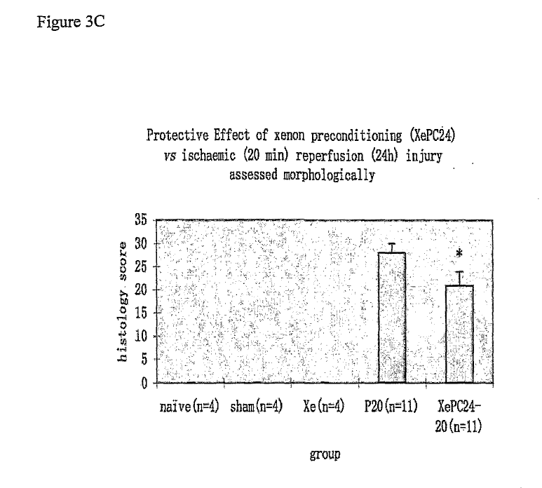

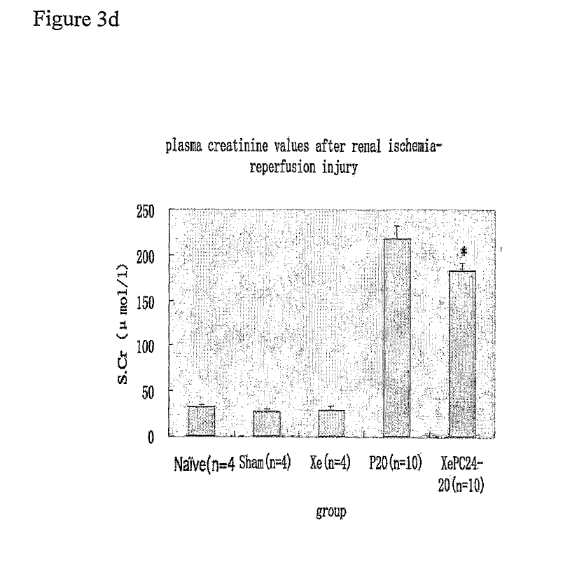

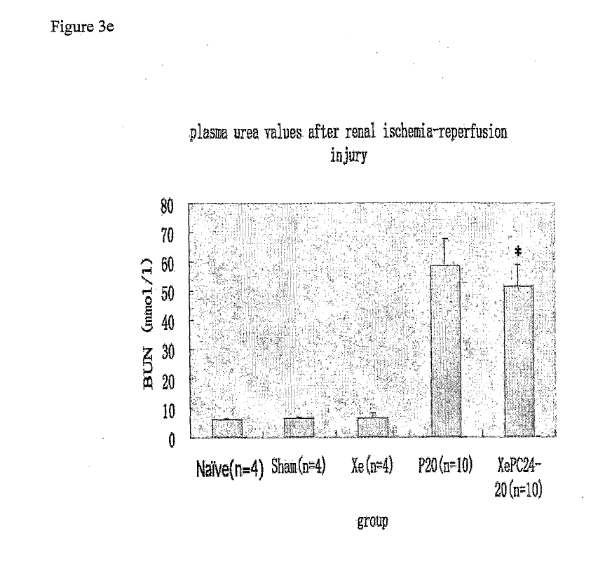

FIG. 3a shows sections of kidneys taken from the mice before said kidney is injured. Said sections have been stained with haematoxyin-eosin (H-E staining—×200 magnification).

FIG. 3b shows sections of kidneys taken from the mice after said kidney has undergone ischaemic-reperfusion (I/R) injury. Said sections have been stained with haematoxyin-eosin (H-E staining—×200 magnification).

FIG. 3c shows that xenon preconditioning (XPD) attenuates renal injury in a renal ischaemia-reperfusion model in adult mice. Renal injury was induced by bilateral renal artery clumping for 20 min 24 hr after which animals were exposed to 75% xenon for 2 hours. The kidneys of said animals were harvested 24 hours after exposure to the xenon. The injuries sustained included loss of nuclei of cells, congestion and dilatation of tubes—these injuries were graded with an arbitrary score of 0 to 3 (0, normal; 1, mild; 2, moderate; 3, severe). A=naïve control; B=ischaemia-reperfusion (IR); C=XPD+IR; D=pathological scoring (mean±SD; n=3).*p<0.05.

FIG. 3d shows the levels of creatinine in blood plasma. S.Cr=serum creatine.

FIG. 3e shows the levels of urea and nitrogen in blood plasma. BUN=blood urea nitrogen.

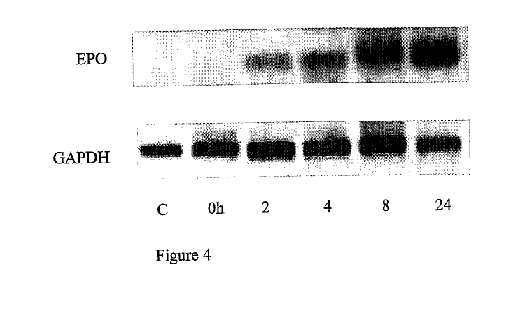

FIG. 4 shows the levels of expression of RNA encoding erythropoietin (EPO), a downstream effector of HIF-1α, and RNA encoding GAPDH, in neonatal rat brains exposed for 2 hours to 75% xenon. Said levels of expression were assessed by quantitative RT-PCR. The brains of said rats were harvested 0-24 hours after exposure to xenon.

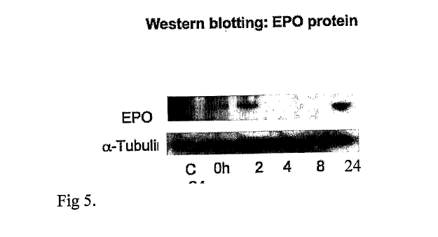

FIG. 5 shows the levels of the polypeptide erythropoietin (EPO), the downstream effector of HIF-1α, and the control polypeptide α-tubulin in neonatal rat brain which were exposed for 2 hours to 75% xenon. Said levels were assessed by western blotting. C=naïve control; 0-24 hr=the time point at which the tissues were harvested after exposure to 75% xenon for 2 hours.

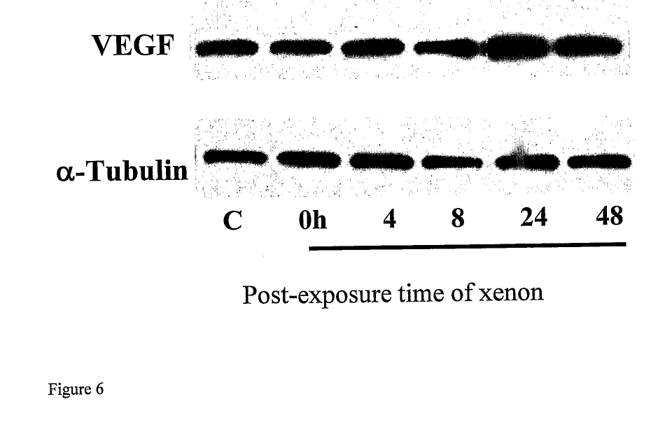

FIG. 6 shows the change over time in the levels of the polypeptide vascular endothelial growth factor (VEGF)—a HIF-1α target gene—and the control polypeptide α-tubulin, in the brains of adult mice which were exposed to xenon for 2 hours. Said brains were analysed by western blotting. C=Control; 0-48 hr=the time point at which tissues were harvested after exposure to 75% xenon for 2 hours.

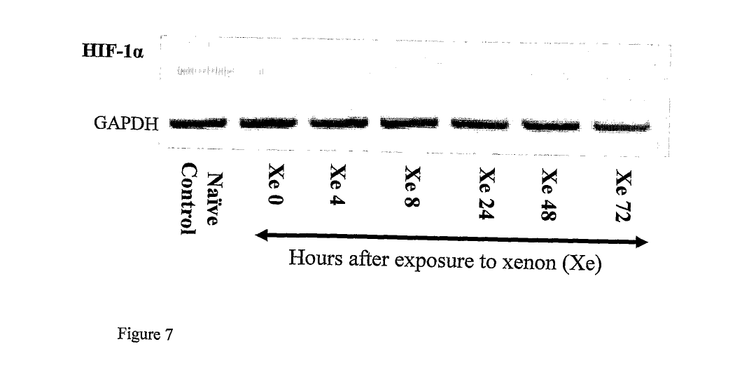

FIG. 7 shows the levels of expression of RNA encoding HIF-1α and RNA encoding GAPDH in adult mice brains exposed for 2 hours to 75% xenon. Said levels of expression were assessed by quantitative RT-PCR. The brains of said mice were harvested 0-72 hours after exposure to xenon.

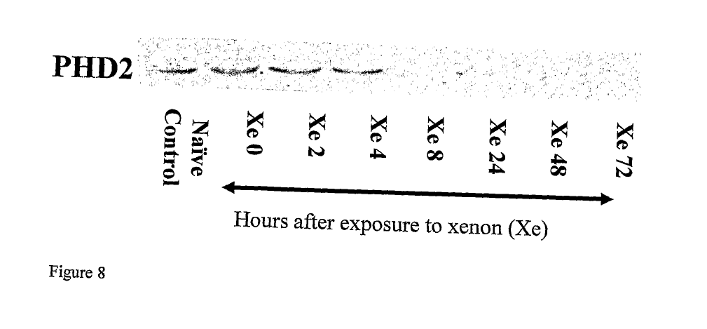

FIG. 8 shows the change over time in the levels of the polypeptide prolyl hydroxylase (PHD2)—an enzyme which has a key role in HIF-1α degradation—in the brains of adult mice which were exposed to xenon for 2 hours. Said brains were analysed by western blotting. 0-48 hr=the time point at which tissues were harvested after exposure to 75% xenon for 2 hours.

FIG. 9 shows the polynucleotide and polypeptide sequences of NM_{—}001530. NM_{—}001530 is a Homo sapiens hypoxia-inducible factor 1, alpha subunit (basic helix-loop-helix transcription factor) (HIF1A).

FIG. 10 shows the polynucleotide and polypeptide sequences of NM_{—}181054. NM_{—}181054 is a Homo sapiens hypoxia-inducible factor 1, alpha subunit (basic helix-loop-helix transcription factor) (HIF1A).

FIG. 11 shows the polynucleotide and polypeptide sequences of NM_{—}001430. NM_{—}001430 is Homo sapiens endothelial PAS domain protein—otherwise known as HIF-2α.

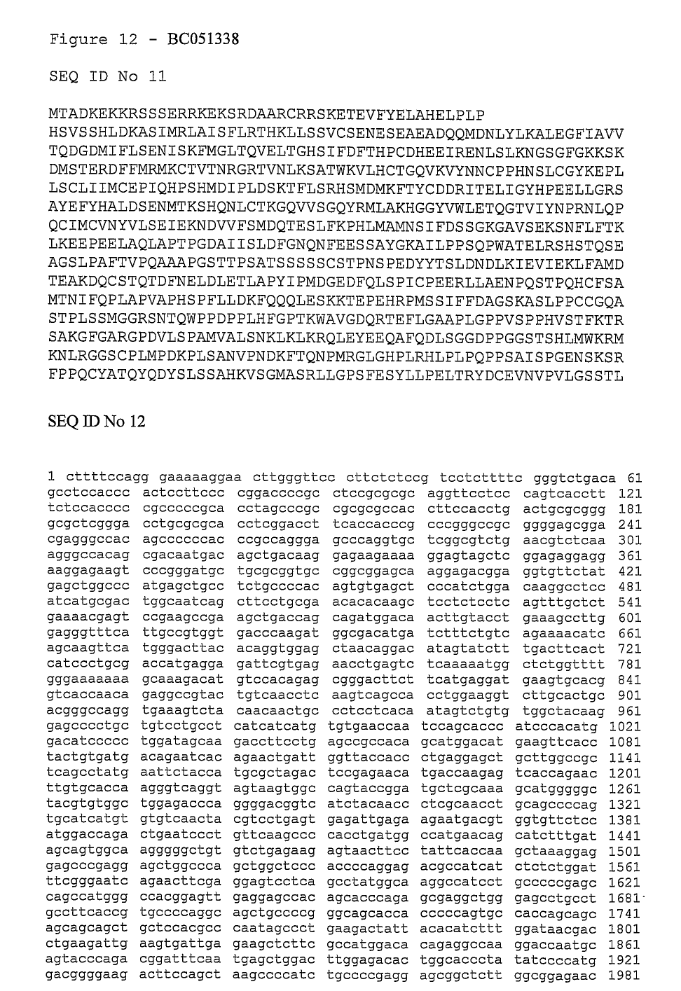

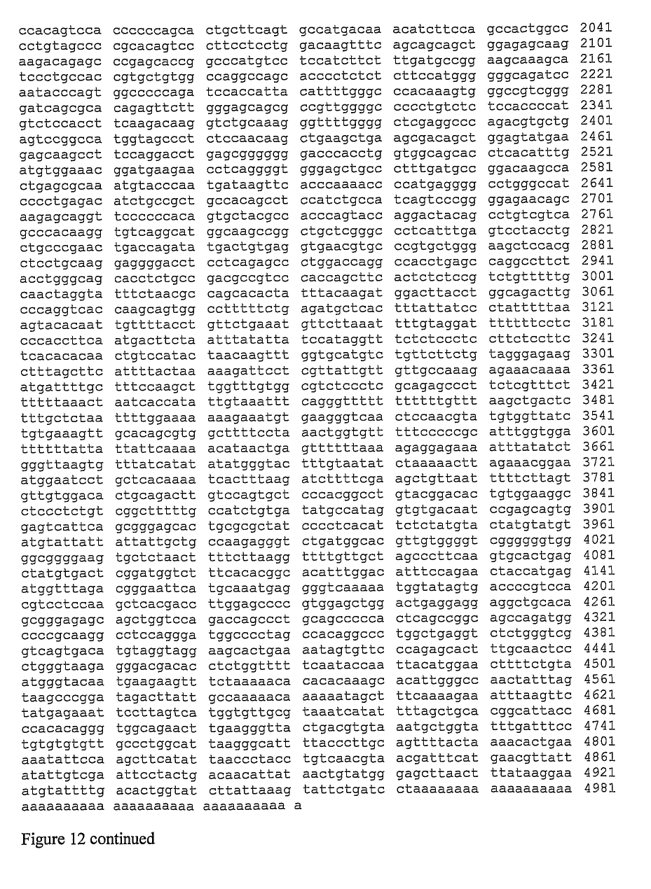

FIG. 12 shows the polynucleotide and polypeptide sequences of BC051338. BC051338 is Homo sapiens endothelial PAS domain protein—otherwise known as HIF-2 α.

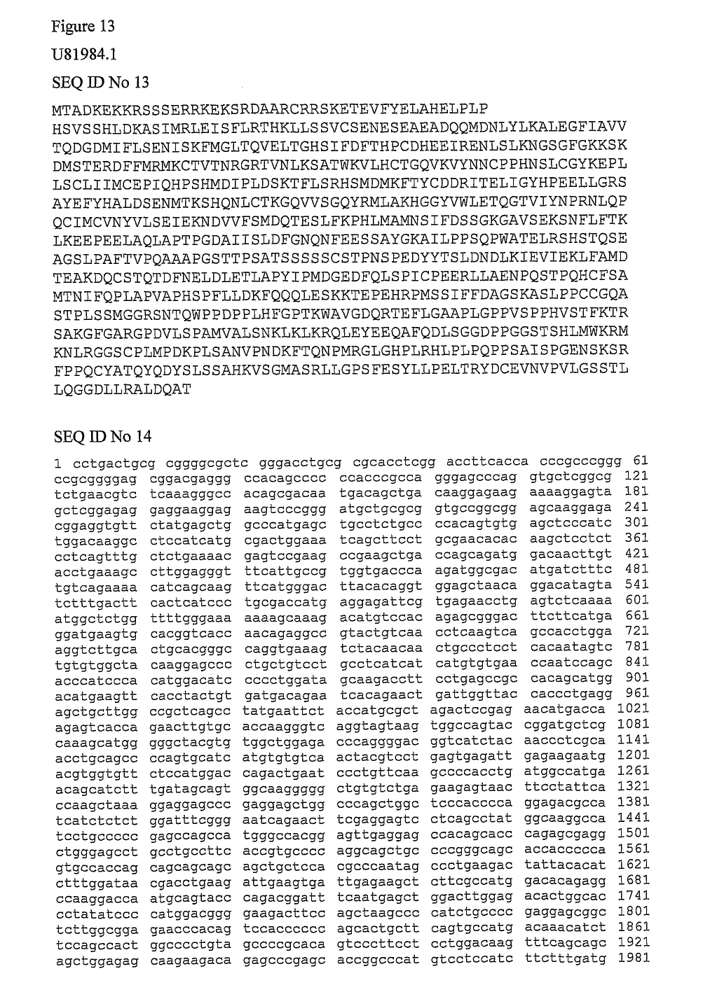

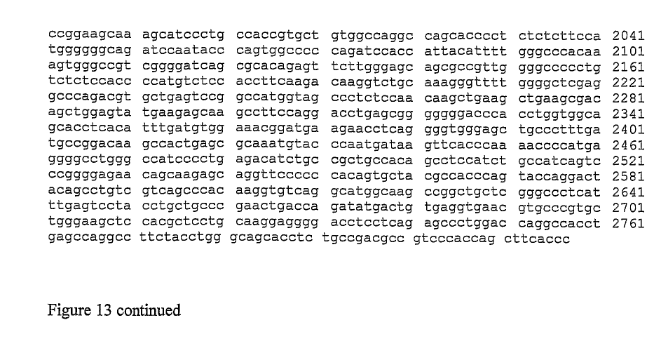

FIG. 13 shows the polynucleotide and polypeptide sequences of U81984.1. U81984.1 is human endothelial PAS domain protein 1 (EPAS1).

### Materials

Monoclonal mouse anti-α-tubulin antibody, monoclonal rabbit anti-NOS antibody were purchased from Sigma, Poole, UK. Polyclonal rabbit anti-BDNF antibody was purchased from Santa Cruz Biotechnology, USA. Monoclonal mouse anti-HIF-1α antibody was purchased from Novus Biologicals, UK. The biotinylated molecular weight ladder and horseradish peroxidase-conjugated goat anti-rabbit and anti-mouse antibodies were purchased from New England Biolab, Hitchin, UK. The nitrocellulose membranes, enhanced chemiluminescence protein detection kit and X-ray films were purchased from Amersham Biosciences, Little Chalfont, UK.

### EXAMPLE 1

### HIF-1α Expression in the Kidney Increases after Exposure to Xenon

### Materials and Methods

### Animals

Studies were performed on adult 8- to 12-wk-old C57BL/6J mice (Jackson Labs, Bar Harbor, Me.) that were fed a standard laboratory diet. All procedures were approved by the Home Office.

The mice were exposed to 75% xenon for two hours. The xenon gas was administered to the mice by inhalation. The kidneys of said mice were then harvested as described below between 0-24 hours after exposure to the xenon gas.

The control mice were not exposed to any gas other than normal ambient air.

The positive control mice were exposed to 8% oxygen for one hour.

### Tissue Homogenisation and Separation of Cytosolic and Membrane Fractions

Once sacrificed, the kidneys were harvested and frozen at −80° C. The frozen tissue was subsequently dissolved in lysis buffer (pH 7.5, 20 mM Tris-HCl, 150 mM NaCl, 1 mM Na₂DTA, 1 mM EGTA, 1% Triton, 2.5 mM sodium pyrophosphate, 1 mM β-glycerophosphate, 1 mM Na₃VO₄, 2 mM DL-dithiothreitol, 1 mM phenylmethanesulfonyl and 1 μg/ml leupeptin) and vigorously homogenised on ice before centrifugation at 3000×g, 4° C. for 10 minutes. Protein concentration in the supernatant was determined by DC (detergent-compatible) protein assay (Bio-Rad, Herts, UK), based on the Lowry method.

### Western Blot

Protein extracts (30 μg per sample) were solubilised in Laemmli sample loading buffer. The samples and a biotinylated molecular weight marker were then denatured at 100° C. for 5 minutes and vortexed for 3 minutes in preparation for SDS-PAGE.

Samples were loaded on a 10.5% SDS electrophoresis gel for protein fractionation by electrophoresis and then electro-transferred to a nitrocellulose membrane.

To eliminate non-specific binding of antibodies, the membrane was incubated for 2½ hours with a blocking solution composed of 5% fat dry milk in Tween-containing Tris buffered saline (TBS-T) (pH 8.0, 10 mM Tris, 150 mM NaCl, 0.1% Tween).

Subsequently, the blocked membrane was incubated overnight at 4° C. with the respective primary antibody at indicated dilutions (Table 1). The primary antibodies were monoclonal mouse anti-α-tubulin antibody, and monoclonal mouse anti-HIF-1α antibody.

TABLE 1

| | |
|---|---|
| Mouse anti-α-tubulin (1:2000) | Anti-mouse antibody (1:1000) |
| Mouse anti-HIF-1α antibody (1:500) | Anti-mouse antibody (1:1000) |

After washing in TBS-T the membrane was incubated with the appropriate goat-derived horseradish peroxidase-conjugated secondary antibody at room temperature for 1½ hours to detect the primary antibodies. The immunoreactive bands were visualised with the enhanced chemiluminescence system and detected on X-ray film. The results were quantified by densitometry as an x-fold increase relative to the control without xenon—the amount of protein applied was normalised by the densitometry of the tubulin (which was unaltered by the intervention itself). The x-fold increase refers to that seen when xenon is replaced by nitrogen or when compared to that present at “0” hours after xenon exposure.

### Results

Exposure to xenon resulted in a time-dependent increase in HIF-1α expression in the kidneys of mice pretreated with xenon (see FIG. 1a). FIG. 1b shows the increase in HIF-1α expression of the mice treated with xenon when compared to the expression of HIF-1α in the control mice.

### EXAMPLE 2

### Xenon Induces HIF-1α Expression in the Same Cells that Xenon Protects from Oxygen-Glucose Deprivation Injury in the Brain

### Materials and Methods

### Animals

Studies were performed on Sprague Dawley, 7 day old rats. All procedures were approved by the Home Office.

The rats were exposed to 75% xenon for two hours. The xenon gas was administered to the rats by inhalation. The brains of said rats were then harvested, as described below, between 0-24 hours after exposure to the xenon gas.

The control rats were not exposed to any gas other than normal ambient air.

### Immunohistochemistry

Paraffin sections (4 μm) were dewaxed in xylene, rehydrated in a series of ethanol washes, and placed in distilled water before staining procedures. Slides were coated with 3-aminopropyl-tri-ethoxysylane.

For detection of HIF isoforms, monoclonal mouse anti-human HIF-1α antibody (67; Novus Biologicals, Littleton, Colo.) and polyclonal rabbit anti-mouse HIF-2α antibodies (PM8 and PM9, obtained from two different rabbits immunised with a peptide containing amino acids 337 to 439 of mouse HIF-2) were used. PM8 and PM9 were provided by Prof. P H Maxwell, Renal Section, Imperial College London, Hammersmith Campus, Du Cane Road, London, W12 ONN. Specific staining of each HIF-isoform was confirmed in immunoblots by using in vitro transcribed and translated mouse HIF-1 and HIF-2 (TnT T7; Promega, Madison, Wis.) and homogenates of rat endothelial cells.

For immunohistochemical analyses, antibody 67 was used at a dilution of 1:6000 and antibodies PM8 and PM9 were used at dilutions of 1:3000.

Detection of bound antibodies was performed by using biotinylated secondary anti-mouse or anti-rabbit antibodies and a catalysed signal amplification system (Dako, Hamburg, Germany) based on the streptavidin-biotin-peroxidase reaction, according to the instructions provided by the manufacturer. Antigen retrieval was performed for 90 seconds in preheated Dako target retrieval solution, using a pressure cooker. All incubations were performed in a humidified chamber. Between incubations, specimens were washed two to four times in buffer (50 mM Tris-HCl, 300 mM NaCl, 0.1% Tween-20, pH 7.6). Control samples included those from air exposed animals, samples prepared with the omission of primary antibodies, and samples prepared with the use of preimmune serum from animals immunised against HIF-2.

### Western Blot

Tissues were homogenised and a western blot was prepared as described in Example 1.

The membrane was incubated with monoclonal mouse anti-α-tubulin antibody (Sigma, Poole, UK) and with monoclonal mouse anti-HIF-1α antibody (Novus Biologicals, UK).

Results

FIG. 2A shows a section of the rat brain taken 6 hours after exposure to xenon. FIG. 2A (i) shows that HIF-1α and HIF-1β expression can be found in the cortex. FIG. 2A (ii) shows the boxed section of FIG. 2A(i) at a higher magnification. As can be seen, HIF-1α expression can be found in the pyramidal cells of the hippocampus.

Exposure to xenon resulted in a time-dependent increase in HIF-1α expression in the brain (see FIG. 2B).

Hence xenon induces expression of HIF-1α and HIF-1β in pyramidal cells in the hippocampus.

### EXAMPLE 3

### Xenon Protects the Kidney from Morphological Damage Induced by Ischaemic-Reperfusion Injury

### Animals

Studies were performed on 8- to 12-wk-old C57BL/6J mice (Jackson Labs, Bar Harbor, Me.) that were fed a standard laboratory diet. All procedures were approved by the Home Office.

The groups of mice were treated as follows:
Naïve=no anaesthesia and no surgery of any kind was carried out on these mice;
Sham=anaesthesia and surgery was carried out on these mice but there was no clamping of the renal pedicle;
Control (or P20)=anaesthesia and surgery together with clamping of the renal pedicle was carried out on these mice but there was but no xenon preconditioning;
XPD group (or XePC24)=mice were exposed to 75% xenon for two hours (XPD=preconditioned with xenon). The xenon gas was administered to the mice by inhalation. Twenty-four hours after this preconditioning the mice then underwent anaesthesia and surgery together with clamping of the renal pedicle; and
Xe=mice were exposed to 75% xenon for two hours but no surgery together with clamping of the renal pedicle was carried out.

### Ischaemic-Reperfusion Renal Injury

Renal injury, as described below, was carried out 24 hours after the mice were exposed to xenon.

Mice were anaesthetised by isoflurane inhalation 2 L/min and placed supine on a heating pad under a warming light, for maintenance of body temperature at 36±1° C. during surgery. Mice were allowed to stabilise for 30 min before they were subjected to bilateral renal pedicles. Through a midline abdominal incision, the left and right renal vessels were occluded with a non-traumatic microvessel clamps for 20 minutes. This duration of ischaemia was chosen, on the basis of earlier preliminary studies in which a reproducible and consistent injury could be produced under these conditions, to maximise the reproducibility of renal injury and to minimise mortality rates for these mice. After both clamps were released, the kidneys showed immediate restoration of renal blood flow excluding the possibility of a vascular thrombus. After unclamping, the incisions were sutured with 5-0 silk. All mice received 0.5 ml saline injected into the open abdomen during surgery to replenish fluid loss. For histological analysis, at the end of 24 hours reperfusion period, the left kidney halves were fixed in 10% formalin solution overnight and embedded in paraffin. Slides were prepared for HE staining (haematoxylin and eosin Staining).

### Histologic Analysis

Kidneys were removed from mice immediately after they were killed, cut in half, fixed in neutral-buffered formalin, and embedded in paraffin. Sections (5 μm thick) of formalin-fixed, paraffin-embedded tissue were mounted on glass slides and stained with haematoxylin-eosin for general histology and quantitative analysis. All tissues were evaluated without investigator knowledge of the group from which it originated.

For quantification of morphologic data, more than 10 low-magnification fields (×200) including both cortex and outer medulla were randomly selected. Renal injury included degeneration (DEG), e.g. loss of nuclei, congestion (CON), dilatation (DIL), was graded with an arbitrary score of 0 to 3: 0, normal; 1, mild; 2, moderate; 3, severe. The total score for each kidney was calculated by addition of all 10 scores (maximum score 30). The histology score was assessed in a blinded manner in each group. Eleven to ten mice were assessed for the control and XPD groups and four mice were assessed for each of the naïve and sham groups.

### Statistical Analyses

Mean±SEM are presented. The significant difference in mean values was evaluated by either a t test or by Dunnett paired t test for multiple comparisons. P<0.05 was considered to be statistically significant.

### Blood Plasma

Blood plasma was harvested from the mice when said mice were sacrificed. This blood plasma was analysed for creatinine and urea nitrogen which are functional markers of renal damage.

### Results:

The data shows that prior exposure to xenon decreased the morphologic injury in the kidney that was produced by 20 min of ischaemia and 24 h of reperfusion (see FIG. 3c).

Furthermore, the results that demonstrate that preconditioning with xenon (i.e. the XePC24 group) significantly decreases the amount of creatinine (μmol) and urea nitrogen (mmol) when compared to animals which have received no preconditioning (i.e. the P20 group)—see FIGS. 3d and 3e.

### EXAMPLE 4

### Xenon Induces the Transcription of Erythropoietin a Downstream Effector of HIF-1α

The aim of this experiment was to visualise the expression level of a downstream effector gene of HIF-1α—i.e. EPO—at different time points after xenon exposure.

### Materials and Methods

### Animals

Neonatal rats (7 days) Sprague Dawley rats were used. All procedures were approved by the Home Office.

The following treatments were carried out:

C=Rat hippocampal brain control stored in −80° C.
0=Rat hippocampal brain treated with xenon for 2 hrs, sacrificed immediately after and stored in −80° C.
2=Rat hippocampal brain treated with xenon for 2 hrs, sacrificed 2 hrs later and stored in −80° C.
4=Rat hippocampal brain treated with xenon for 2 hrs, sacrificed 4 hrs later and stored in −80° C.
8=Rat hippocampal brain treated with xenon for 2 hrs, sacrificed 8 hrs later and stored in −80° C.
24=Rat hippocampal brain treated with xenon for 2 hrs, sacrificed 24 hrs later and stored in −80° C.
P2=Rat hippocampal brain positive control with ischaemia injury for 45 mins, harvested after 24 hrs, stored in −80° C.

### RNA Extraction

Total RNA was extracted from neonatal rat brains.

The followings are the reagents and equipment were used for RNA extraction.

TABLE 2a

| | Company | Category | |
|---|---|---|---|
| Product | Name | No. | Other Info |
| RNA later (100 ml) | Sigma | R0901 | |
| RNase Erase | Q-Biogene | 2440-204 | |
| Ethanol (absolute) Biology | BDH | 437433T | MW = 46.07 g/mol |
| Grade | | | Flammable |
| RNeasy Mini Kit (50): | Qiagen | 74104 | |
| 50 RNeasy Mini Spin | | | |
| Columns, Collection Tubes | | | |
| (1.5 ml and 2 ml), RNase | | | |
| Free Reagents and Buffers | | | |
| (RLT, RW1, RPE and | | | |
| RNase-free water) | | | |

### Reagents for Reverse Transcription

Reverse transcription of the RNA in order to obtain the first-strand cDNA was carried out using techniques well known in the art. Table 2 details the reagents which were used for reverse transcription.

TABLE 2b

| Product | Company | Cat No. | Storage |
|---|---|---|---|
| Random Oligo (dT) Primer | Promega | C1101 | −20° C. |
| [20 μg], 500 μg/mol | | | |
| SUPERase-In [20 U/μl, | Ambion | 2694 | −20° C. |
| 2500U] | | | |
| SuperScriptII Reverse | Invitrogen | 18064022 | −20° C. |
| Transcriptase [200 U/μl] | | | |
| DTT | | | |
| 5x First Strand Buffer [1 ml] | | | |
| PCR nucleotide mix | Promega | C1141 | −20° C. |
| [200 μl. 10 mM] | | | |

### PCR Amplification

PCR amplification was carried out using techniques well known in the art. Table 3 details the reagents used for PCR amplification.

TABLE 3
Reagents for PCR

| Product | Company | Cat No. | Storage |
|---|---|---|---|
| PCR nucleotide mix [200 μl. 10 mM] | Promega | C1141 | −20° C. |
| 1. Taq DNA Polymerase in | Promega | M1661 | −20° C. |
| Storage B [100 μg, 5 μ/μl] | | | |
| 2. Taq DNA Polymerase 10x | | | |
| Reaction Buffer without | | | |
| MgCl2 (1.2 ml) | | | |
| Magnesium Chloride [25 mM, 750 μl] | | | |

The primers used in the PCR amplification were:

(SEQ ID No 1)

GAPDH forward primer 5′- ACCCATCACCATCTTCCA -3′

(SEQ ID No 2)

GAPDH reverse primer 5′- CATCACGCCACAGCTTTCC -3′

(SEQ ID No 3)

EPO forward primer 5′- AGTCGCGTTCTGGAGAGGTA -3′

(SEQ ID No 4)

EPO reverse primer 5′- AGGATGGCTTCTGAGAGCAG -3′

Reverse Transcription (RT) and PCR amplification
i) The samples as described in Table 4 were used for reverse transcription part I. The components of Part I reverse transcription reaction are listed in Table 4.

TABLE 4
Components for Part I reverse transcription

| | | Rat | Rat | Rat | Rat | Rat |
|---|---|---|---|---|---|---|
| | Rat | Brain | Brain | Brain | Brain | Brain |
| | Brain | Xenon | Xenon | Xenon | Xenon | Xenon |
| Chemical | Control | 0 hr | 2 hr | 4 hr | 8 hr | 24 hr |
| Concentration | 271.8 | 79.4 | 166.5 | 147.4 | 419.2 | 157.8 |
| of total RNA | | | | | | |
| (ng/μl) | | | | | | |
| RNA (μl) | 2.92 | 10 | 4.77 | 5.39 | 1.89 | 5.03 |
| Total amount | 793.7 | 794 | 794.2 | 794.5 | 792.3 | 793.7 |
| of RNA (ng) | | | | | | |
| Oligo (dT) | 1 | 1 | 1 | 1 | 1 | 1 |
| primer | | | | | | |
| (500 μg/ml) | | | | | | |
| dNTP mix | 1 | 1 | 1 | 1 | 1 | 1 |
| (10 mM each) | | | | | | |

Water was added to bring the total volume to 12 μl

The reverse transcription mixture was heated to 65° C. for 5 min before being cooled on ice for 1 min. Then, the following components (see Table 5) were added into each tube for reverse transcription Part II.

TABLE 5
Components for reverse transcription part II

| Chemical | Volume (μl) |
|---|---|
| Superasein (2 μg/μl) | 1 |
| 5X First-Strand Buffer | 4 |
| 0.1 M DTT | 2 |
| Total vol in the PCR tube | 19 |

The mixture was then incubated at 42° C. for 2 minutes. Two 2 minutes later, 1 μl (200 units) of SuperScript II (Invitrogen) was added and mixed by pippetting (total volume was 20 μl in each tube).

The tubes were then incubated at 42° C. for 50 min. Followed by inactivation by heating at 70° C. for 15 min.

The following cDNA samples and primer pairs were used for PCR amplification (see Table 6 and Table 7).

TABLE 6

| | Tube no. | | | | | |
|---|---|---|---|---|---|---|
| | 1 | 2 | 3 | 4 | 5 | 6 |
| Tissue Sample | Rat | Rat | Rat | Rat | Rat | Rat |
| | Brain | Brain | Brain | Brain | Brain | Brain |
| | Control | 0 hr | 2 hr | 4 hr | 8 hr | 24 hr |

| | |
|---|---|
| Primer pairs | EPO forward and reverse primers (SEQ ID Nos 3 and 4) |

TABLE 7

Tube no.

| | 7 | 8 | 9 | 10 | 11 | 12 |
|---|---|---|---|---|---|---|
| Tissue | Rat | Rat | Rat | Rat | Rat | Rat |
| Sample | Brain | Brain | Brain | Brain | Brain | Brain |
| | Control | 0 hr | 2 hr | 4 hr | 6 hr | 8 hr |

| | |
|---|---|
| Primer | EPO forward and reverse primers |
| pairs | (SEQ ID Nos 3 and 4) & |
| | GAPDH forward and reverse primers |
| | (SEQ ID Nos 1 and 2) |

The following components (see Table 7) were then added into the PCR tubes.

TABLE 13
The reagents contained in each PCR tube

Tube no.

| | |
|---|---|
| 1-6 | 7-12 |

| | |
|---|---|
| Vol of cDNA (μl) | 2 |
| MgCl2 25 mM (μl) | 6 |
| final conc 3 mM | |
| 10x reaction buffer | 5 |
| dNTP mix (10 mM each) | 1 |
| final conc 800 μM | |

| | | |
|---|---|---|
| GAPDH Forward primer (20 μM) | 0 | 0.25 |
| final conc 0.1 μM | | |
| GADPH Reverse primer (20 μM) | 0 | 0.25 |
| final conc 0.1 μM | | |
| EPO Forward primer (20 μM) | 2.5 | 2.5 |
| final conc 1 μM | | |
| EPO Reverse primer (20 μM) | 2.5 | 2.5 |
| final conc 1 μM | | |
| Volume of water needed to make | 31 | 30.5 |
| up 50 μl | | |

Prior to PCR amplification the samples were incubated for 3 minutes at 96° C. before 0.5 μl of Taq DNA polymerase was added to each tube.

PCR amplification was carried out using the following conditions: denaturation at 96° C. for 30 seconds, followed 60° C. for 1 minute for primer annealing and 72° C. for 3 minutes for extension. A total of 30 PCR amplification cycles were used before a final extension at 72° C. for 7 minutes followed by storage at 4° C.

The resulting PCR amplification products were electrophoresed on a 1×TAE agarose gel in 1×TAE buffer and visualised using Fluor-S MultiImager BIORAD. Nucleotides intercalating with ethidium bromide fluoresce under UV light. As the level of fluorescence is approximately proportional to the amount of intercalated ethidium bromide, the abundance of amplified DNA within samples can be compared; in other words, the level of expression of a gene of interest can be determined. The Fluor-S MultiImager contains a UV light box for visualisation of the DNA bands and a photograph is taken by the machine to store the image data.

### Results

Exposure to xenon results in a time-dependent increase in EPO expression (a downstream effector of HIF-1α) in the brains of neonatal rats pretreated with xenon (see FIG. 4).

As a control, the expression of GAPDH was monitored.

### EXAMPLE 5

### Xenon Induces the Transcription of Erythropoietin, a Downstream Effector of HIF-1α

### Animals

Sprague Dawley, 7 day neonatal rats were used. All procedures were approved by the Home Office.

The neonatal rats were exposed to 75% xenon for two hours. The gas was administered by gas inhalation. The brains of said rats were then harvested as described below between 0-24 hours after exposure to the xenon gas.

The control neonatal rats were not exposed to any gas other than normal ambient air.

### Methods

The tissues were homogenised and western blots of neonatal rat brain samples were carried out as described in Example 1.

The primary antibodies were anti-α-tubulin antibody, and monoclonal mouse anti-erythropoietin antibody.

### Results

Exposure to xenon results in a time-dependent increase in the expression of EPO protein (the downstream effector of HIF-1α) in the brains of neonatal rats pretreated with xenon (see FIG. 5).

### EXAMPLE 6

### Synergy is Observed when Xenon and Cooling Administered to the Brain

### Animals

Sprague Dawley, 7 day neonatal rats were used. All procedures were approved by the Home Office.

The neonatal rats were exposed to:
(i) 20% xenon alone for 2 hours;
(ii) 35° C. hypothermia for 90 minutes; or
(iii) Hypothermia (35° C.) for 90 minutes before, 1 hour later, 20% xenon for 2 hours.

The gas was administered by gas inhalation.

The brains of said rats were then harvested at a set time after exposure to the xenon gas. A total of 11 rats were examined for each treatment group.

The brains were assessed for cortical infarction in the affected hemisphere.

The “control” rats did not undergo an ischaemic injury to the brain. The “intervention” rats under went an ischaemic injury to the brain.

### Results

TABLE 14
Total area of infraction.

| Group | 35° C. Hypothermia | 20% xenon alone | Combination |
|---|---|---|---|
| Control | 88.6 (±7.7) | 88.3 (±4.1) | 92.7 (±6.7) |
| Intervention | 93.7 (±0.8) | 85.3 (±8.9) | 74.1 (±7.8)* |

*p = <0.05

The table 14 shows the total area of infraction in the brains of the rats.

As can be seen, rats treated with xenon alone showed a lower area of infraction in the brain than rats treated with hypothermia alone. However rats treated with hypothermia followed by xenon showed an even lower area of infraction in the brain than rats treated with hypothermia alone or xenon alone.

Hence there is synergy when rats are treated with xenon and cooling—even when the treatment is administered asynchronously.

### EXAMPLE 7

### Increase in the Expression of VEGF a HIF-1α Target Gene by Xenon Pre-Conditioning

### Materials and Methods

### Animals

Studies were performed on 8 to 12-wk-old C57BL/6J mice (Jackson Labs, Bar Harbor, Me.) that were fed a standard laboratory diet. All procedures were approved by the Home Office.

The mice were exposed to 75% xenon and 25% oxygen for two hours. The xenon gas was administered to the mice by inhalation. The brains of said adult mice were then harvested as described below between 0-48 hours after exposure to the xenon gas.

The control mice were not exposed to any gas other than normal ambient air.

### Methods

The tissues were homogenised and western blots of adult mice brain samples were carried out as described in Example 1.

The primary antibodies were anti-α-tubulin antibody, and monoclonal mouse anti-VEGF antibody.

The anti-α-tubulin antibody was the control.

### Results

The data shows that exposure to xenon (i.e. preconditioning with xenon) resulted in a time-dependent increase in the expression of VEGF—a HIF-1α target gene (see FIG. 6).

### EXAMPLE 8

### The Expression of HIF-1α Gene is not Modulated by Xenon Preconditioning

### Materials and Methods

### Animals

Studies were performed on 8- to 12-wk-old C57BL/6J mice (Jackson Labs, Bar Harbor, Me.) that were fed a standard laboratory diet. All procedures were approved by the Home Office.

The mice were exposed to 75% xenon and 25% oxygen for two hours. The xenon gas was administered to the mice by inhalation. The brains of said adult mice were then harvested as described below between 0-72 hours after exposure to the xenon gas.

The naïve control mice were not exposed to any gas other than normal ambient air.

### Methods

Total RNA was extracted as described in Example 4. In addition RT-PCR was carried out as described in Example 4. The primers used for the PCR amplifications were:

(SEQ ID No 1)

GAPDH forward primer 5′- ACCCATCACCATCTTCCA -3′

(SEQ ID No 2)

GAPDH reverse primer 5′- CATCACGCCACAGCTTTCC -3′

(SEQ ID No 15)

HIF-1α forward primer 5′- TCA AGT CAG CAA
CGT GGA AG -3′

(SEQ ID No 16)

HIF-1α reverse primer 5′- TAT CGA GGC TGG
GTC GAC TG -3′

### Results

The data shows that exposure to xenon does not modulate the transcription of the HIF-1α gene over time (see FIG. 7).

As a control, the expression of GAPDH was monitored (see FIG. 7).

### EXAMPLE 9

### Xenon Preconditioning Reduces the Transcription of PHD2—an Upstream Degrader of HIF-1α

### Materials and Methods

### Animals

Studies were performed on 8- to 12-wk-old C57BL/6J mice (Jackson Labs, Bar Harbor, Me.) that were fed a standard laboratory diet. All procedures were approved by the Home Office.

The mice were exposed to 75% xenon and 25% oxygen for two hours. The xenon gas was administered to the mice by inhalation. The brains of said adult mice were then harvested as described below between 0-48 hours after exposure to the xenon gas.

The naïve control mice were not exposed to any gas other than normal ambient air.

### Methods

The tissues were homogenised and western blots of adult mice brain samples were carried out as described in Example 1.

The primary antibody was monoclonal mouse anti-PHD2 antibody.

### Results

The data shows that exposure to xenon (i.e. preconditioning with xenon) resulted in a time-dependent reduction in the expression of PHD2 (see FIG. 8). PHD2 is an enzyme which is vital to HIF-1α degradation.

Without wishing to be bound by theory, xenon-induced HIF expression (such as (HIF-1α expression) may be due, at least in part, to decreased degradation of HIF by PHD2.

All publications mentioned in the above specification are herein incorporated by reference. Various modifications and variations to the present invention will be apparent to those skilled in the art without departing from the scope and spirit of the present invention. Although the present invention has been described in connection with specific preferred embodiments, it should be understood that the invention as claimed should not be unduly limited to such specific embodiments.

1-27. (canceled)

28. A method of protecting an organ, tissue or cell from injury, wherein said organ, tissue or cell expresses hypoxia inducible factor (HIF), said method comprising administering to the organ, tissue or cell xenon as the sole organ, tissue or cell protectant or a pharmaceutical composition comprising xenon as the sole organ, tissue or cell protectant, wherein said organ, tissue or cell is not derived from any of brain, heart, embryonic nigral tissue, liver, lung, cornea, neurons or intestinal epithelial cells.

29. The method of claim 28 wherein said organ, tissue or cell is selected from the group consisting of kidney, pancreas, a reproductive organ, a reproductive tissue, muscle, skin, fat, a fertilized embryo and a joint.

30. The method of claim 29 wherein said organ is kidney or said tissue is kidney tissue.

31. The method of claim 28 wherein said organ, tissue or cell is an ex vivo organ, tissue or cell.

32. The method of claim 28 wherein said organ, tissue or cell is an in vivo organ, tissue or cell.

33. A method of reducing the expression of a upstream degrader of HIF or inducing the expression of HIF or inducing the expression of a downstream effector of HIF in an organ, tissue or cell, wherein said method comprises administering xenon or a pharmaceutical composition thereof to said organ, tissue or cell, and wherein said organ, tissue or cell is not derived from brain, heart, embryonic nigral tissue, liver, lung, cornea, neurons or intestinal endothelial cells.

34. The method of claim 33 wherein the upstream degrader of HIF is prolyl hydroxylase 2 (PHD2).

35. The method of claim 33 wherein the downstream effector is erythropoietin.

36. The method of claim 33 wherein said organ, tissue or cell is selected from the group consisting of kidney, pancreas, a reproductive organ, a reproductive tissue, muscle, skin, fat, a fertilized embryo and a joint.

37. The method of claim 36 wherein said organ is kidney.

38. The method of claim 33 wherein the xenon or pharmaceutical composition thereof is the sole organ, tissue or cell protectant that is administered.

39. The method of claim 28 wherein the xenon or pharmaceutical composition comprising xenon is administered before the organ, tissue or cell is injured.

40. The method of claim 28 further comprising one or more steps selected from:
(i) cooling the organ, tissue or cell;
(ii) perfusing and/or suprefusing the organ, tissue or cell with an agent that supplies energy to organ, tissue or cell; and
(iii) perfusing and/or superfusing the organ, tissue or cell with an agent that decreases the energy requirements of the organ, tissue or cell;
wherein the steps (i) to (iii) are conducted when the organ, tissue or cell is injured.

41. The method of claim 33 further comprising one or more steps selected from:
(i) cooling the organ, tissue or cell;
(ii) perfusing and/or suprefusing the organ, tissue or cell with an agent that supplies energy to organ, tissue or cell; and
(iii) perfusing and/or superfusing the organ, tissue or cell with an agent that decreases the energy requirements of the organ, tissue or cell;
wherein the steps (i) to (iii) are conducted when the organ, tissue or cell is injured.

42. The method of claim 28 further comprising one or more steps selected from:
(i) cooling the organ, tissue or cell;
(ii) perfusing and/or suprefusing the organ, tissue or cell with an agent that supplies energy to organ, tissue or cell; and
(iii) perfusing and/or superfusing the organ, tissue or cell with an agent that decreases the energy requirements of the organ, tissue or cell;
wherein the steps (i) to (iii) are conducted before the organ, tissue or cell is injured.

43. The method of claim 33 further comprising one or more steps selected from:
(i) cooling the organ, tissue or cell;
(ii) perfusing and/or suprefusing the organ, tissue or cell with an agent that supplies energy to organ, tissue or cell; and
(iii) perfusing and/or superfusing the organ, tissue or cell with an agent that decreases the energy requirements of the organ, tissue or cell;
wherein the steps (i) to (iii) are conducted before the organ, tissue or cell is injured.

44. The method of claim 28 further comprising one or more steps selected from:
(i) administering a chelator and/or a converter of a reactive oxygen species;
(ii) administering an agent that decreases the level of cytokines and/or chemokines;
(iii) cooling the organ, tissue or cell; and
(iv) decreasing the energy requirements of the organ, tissue or cell;
Wherein steps (i) to (iv) are conducted after the cell, tissue or organ is injured.

45. The method of claim 33 further comprising one or more steps selected from:
(i) administering a chelator and/or a converter of a reactive oxygen species;
(ii) administering an agent that decreases the level of cytokines and/or chemokines;
(iii) cooling the organ, tissue or cell; and
(iv) decreasing the energy requirements of the organ, tissue or cell;
Wherein steps (i) to (iv) are conducted after the cell, tissue or organ is injured.

46. The method of claim 32 wherein the organ is kidney or the tissue is kidney tissue.

47. The method of claim 46 further comprising one or more steps selected from:
(i) increasing the flow of urine from a subject; and
(ii) performing dialysis;
Wherein steps (i) to (ii) are conducted after said organ, tissue or cell is injured.

48. The method of claim 28 wherein the xenon or pharmaceutical composition comprising xenon is administered before or after the organ, tissue or cell is cooled.

49. A method of delivering an HIF activator to an organ, tissue or cell wherein said HIF activator is administered before or after the organ, tissue or cell is cooled.

50. The method of claim 49 wherein said organ is brain or said tissue is brain tissue or said cell is a brain cell.

51. The method of claim 28 further comprising the administration of a vector comprising an HIF responsive element.

52. The method of claim 51 wherein the vector comprises a polynucleotide sequence capable of expressing a suicide gene, wherein said polynucleotide gene is operably linked to an HIF responsive element.

53. The method of claim 33 further comprising the administration of a vector comprising an HIF responsive element.

54. The method of claim 53 wherein the vector comprises a polynucleotide sequence capable of expressing a suicide gene, wherein said polynucleotide gene is operably linked to an HIF responsive element.
